(19) United States
(12) Patent Application Publication
(10) Pub. No.: US 2026/0263454 A1
(43) Pub. Date: Sep. 10, 2026

(54) USES OF A CHECKPOINT KINASE 1 (CHK1) INHIBITOR

(71) Applicant: Boundless Bio, Inc., San Diego, CA (US)

(72) Inventors: Christian HASSIG, San Diego, CA (US); Klaus Wolfgang WAGNER, San Diego, CA (US); Shailaja KASIBHATLA, San Diego, CA (US); Julius Lim APUY, San Diego, CA (US); Anthony B. PINKERTON, Rancho Santa Fe, CA (US); James Jisub LEE, San Diego, CA (US)

(21) Appl. No.: 19/166,343

(22) PCT Filed: Mar. 19, 2024
(86) PCT No.: PCT/US2024/020515

§ 371 Date: Sep. 17, 2025

Related U.S. Application Data
(60) Provisional application No. 63/491,198, filed on Mar. 20, 2023.
(60) Provisional application No. 63/591,370, filed on Oct. 18, 2023.
(60) Provisional application No. 63/623,663, filed on Jan. 22, 2024.
(60) Provisional application No. 63/557,804, filed on Feb. 26, 2024.

(51) Int. Cl. A61K 31/497; A61K 31/506; A61K 31/517; A61K 31/519; A61K 31/5377; A61P 35/00; A61P 35/04; C12Q 1/6841; C12Q 1/6886

(52) U.S. Cl. CPC A61K 31/497; A61K 31/506; A61K 31/517; A61K 31/519; A61K 31/5377; A61P 35/00; A61P 35/04; C12Q 1/6841; C12Q 1/6886; C12Q 2600/156

(57) ABSTRACT

Provided herein are methods for the treatment of cancer. The methods include administering to a subject in need a therapeutically effective amount of a Chk1 inhibitor disclosed herein alone or in in combination with an additional therapeutic agent.

### CROSS-REFERENCE

This application claims the benefit of U.S. Provisional Application Ser. No. 63/491,198 filed Mar. 20, 2023; U.S. Provisional Application Ser. No. 63/591,370 filed Oct. 18, 2023; U.S. Provisional Application Ser. No. 63/623,663 filed Jan. 22, 2024; and U.S. Provisional Application Ser. No. 63/557,804 filed Feb. 26, 2024; which are hereby incorporated by reference in their entirety.

### BACKGROUND OF THE INVENTION

Cancer remains the second leading cause of death in the United States (US), accounting for approximately 1,900,000 new diagnoses and 610,000 deaths on an annual basis. Globally, cancer is the second leading cause of death, and was responsible for nearly 10 million deaths in 2020; nearly 1 in 6 deaths was due to cancer. The number of new cases is expected to rise by 70% over the next 2 decades.

In solid malignancies, metastatic spread, and systemic disease accounts for approximately 90% of cancer-related deaths. Despite progress made over the past few decades, there remains a need for the development of targeted interventions for advanced or metastatic solid tumors.

Patients with cancers that harbor oncogene amplifications, with the exception of HER2 (or ERBB2), have no targeted therapies approved as standard of care. These patients suffer worse survival rates than patients with other forms of oncogene alteration or with no known oncogene alteration. Focal high-copy number oncogene amplifications are frequently observed to occur on extrachromosomal DNA (ecDNA). ecDNA are cancer-specific circular fragments of genomic DNA that encode full length genes and regulatory regions such as promoters. ecDNA are physically distinct from chromosomes and are engendered with unique properties, including an open chromatin architecture associated with hyper-transcription and a predilection for structural variation. In addition, because ecDNA lack centromeres, they are inherited during cellular division via acentric, non-Mendelian segregation, which enables high copy number gene heterogeneity across the tumor cell population. Due to these properties, ecDNA are a common cellular mechanism for oncogene amplification (e.g., EGFR), and they facilitate hyper-transcription and overexpression of oncoproteins, which drive tumor growth and survival. Moreover, these features afford oncogene amplified ecDNA-enabled tumor cells with unparalleled genomic plasticity, facilitating both oncogenesis and circumvention of therapeutic pressure through rapid genomic evolution. Cancer cells that harbor oncogene amplifications on ecDNA bear high levels of intrinsic DNA replication stress (RS). The checkpoint kinase 1 (Chk1) serves an essential role in managing RS, making Chk1a potential therapeutic target for cancers that have intrinsic elevated RS, including those with ecDNA-enabled oncogenic amplifications. Consistent with this hypothesis, ecDNA-enabled oncogene-amplified tumor cells display enhanced sensitivity to Chk1 inhibition when compared to ecDNA negative non-amplified cells. Applying targeted therapy (e.g., EGFR inhibitor) pressure to the protein products of oncogenes (e.g., EGFR) amplified on ecDNA induces cancer cells to evade such pressure, and these resistance mechanisms further increase RS and reliance upon Chk1. Accordingly, combining targeted therapy pressure (e.g., EGFR inhibitor) with Chk1 pressure (i.e., Chk1 inhibitor) in ecDNA-enabled tumor cells provides a synergistic therapeutic effect.

There are currently no therapies approved to treat patients with the aforementioned oncogene-amplified tumors.

### BRIEF SUMMARY OF THE INVENTION

Disclosed herein is a method of treating cancer in a subject in need thereof comprising administering to the subject a pharmaceutical composition comprising 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

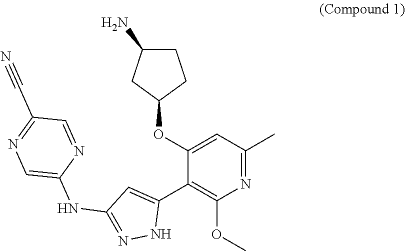 [SMILES: COc1nc(C)cc(O[C@@H]2CC[C@H](N)C2)c1-c1cc(Nc2cnc(C#N)cn2)n[nH]1]

or a pharmaceutically acceptable salt thereof, at a dose of about 10 mg to about 400 mg, whereby the subject experiences a therapeutic response.

In some embodiments of a method disclosed herein, the dose of Compound 1 is between about 10 mg to about 20 mg, about 20 mg to about 40 mg, about 40 mg to about 80 mg, about 80 mg to about 120 mg, about 120 mg to about 160 mg, about 160 mg to about 200 mg, about 200 mg to about 240 mg, about 200 mg to about 400 mg.

In some embodiments of a method disclosed herein, the dose of Compound 1 is 10 mg, about 20 mg, about 40 mg, about 80 mg, about 120 mg, about 160 mg, about 200 mg, or about 240 mg.

In some embodiments of a method disclosed herein, the cancer comprises a solid tumor.

In some embodiments of a method disclosed herein, the cancer comprises a locally advanced or metastatic non-resectable solid tumor.

In some embodiments of a method disclosed herein, the cancer comprises a tumor or tumor cells harboring an oncogene amplification.

In some embodiments of a method disclosed herein, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FGFR4, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments of a method disclosed herein, the oncogene amplification resides on ecDNA.

In some embodiments of a method disclosed herein, the oncogene amplification resides on one or more chromosomal loci.

In some embodiments of a method disclosed herein, the oncogene amplification is an ecDNA-derived amplification.

In some embodiments of a method disclosed herein, the oncogene amplification has a copy number of at least 6, at least 8, at least 10, at least 15, at least 20, or more than 20 copies of the oncogene or portion thereof.

In some embodiments of a method disclosed herein, the subject has undergone one or more prior therapies.

In some embodiments of a method disclosed herein, the subject was non-responsive to the one or more prior therapies.

In some embodiments of a method disclosed herein, the subject developed resistance to the one or more prior therapies.

In some embodiments of a method disclosed herein, the composition is administered orally.

In some embodiments of a method disclosed herein, the composition is administered every other day.

In some embodiments of a method disclosed herein, the composition is administered on a cycle of day 1 and day 3 followed by a 4 day dosing holiday.

In some embodiments of a method disclosed herein, the composition is administered every 3 days or weekly.

In some embodiments of a method disclosed herein, the composition is administered with a dosing holiday of 4 days, 4-7 days, 5 day, 7 days, 11 days, or 14 days.

In some embodiments of a method disclosed herein, the composition is administered on a cycle of day 1 and day 2 followed by a 5 day dosing holiday.

In some embodiments of a method disclosed herein, the composition is administered once weekly.

In some embodiments of a method disclosed herein, the composition is administered on a cycle of days 1, 2 and 3, followed by an 11 day dosing holiday.

In some embodiments of a method disclosed herein, the dose of Compound 1 about 80 mg, about 120 mg, about 160 mg, about 200 mg, or about 240 mg, and the composition is administered on a cycle of day 1 and day 2 followed by a 5 day dosing holiday.

In some embodiments of a method disclosed herein, the dose of Compound 1 about 80 mg, about 120 mg, about 160 mg, about 200 mg, or about 240 mg, and the composition is administered once weekly.

In some embodiments of a method disclosed herein, the cancer is bladder cancer, breast cancer, endometrial cancer, esophageal cancer, gastric cancer, head and neck squamous cell carcinoma, liposarcoma, non-small cell lung adenocarcinoma, non-small cell lung cancer, non-small cell squamous lung cancer, or ovarian cancer.

In some embodiments of a method disclosed herein, the cancer is an ovarian cancer.

In some embodiments of a method disclosed herein, the ovarian cancer is a platinum resistant high-grade serous ovarian cancer, a primary peritoneal cancer, or a fallopian tube cancer.

In some embodiments of a method disclosed herein, the cancer is a uterine cancer.

In some embodiments of a method disclosed herein, the uterine cancer is a high-grade endometrial carcinoma, a uterine serous carcinoma or a uterine carcinosarcoma.

In some embodiments of a method disclosed herein, the cancer is a neuroblastoma.

In some embodiments of a method disclosed herein, the treatment further comprises administering an additional therapeutic agent.

In some embodiments of a method disclosed herein, the oncogene amplification comprises a CDK4, CDK6, EGFR, FGFR1, FGFR2, or FGFR3 amplification.

In some embodiments of a method disclosed herein, the oncogene amplification comprises a wildtype FGFR1, FGFR2, FGFR3, or FGFR4 amplification.

In some embodiments of a method disclosed herein, the oncogene amplification comprises a wildtype EGFR amplification.

In some embodiments of a method disclosed herein, the oncogene amplification comprises an amplification of EGFR and wherein the treatment further comprises administering an EGFR inhibitor.

In some embodiments of a method disclosed herein, the application of EGFR is an amplification of a wildtype EGFR.

In some embodiments of a method disclosed herein, the EGFR inhibitor is selected from the group consisting of abivertinib, ABX-900, afatinib, agerafenib (RXDX-105), alflutinib mesylate, amivantamab, APL-1898, ASK-120067, aumolertinib (almonertinib), BBT-176, BDTX-1535, BDTX-189, BEBT-109, befortinib mesylate, beitatini, BLU-701, BLU-945, BPI-361175, BPI-7711, BPI-D0316, C-005, CDP1, cetuximab, CH-7233163, CK-101, CMAB-017, dacomitinib, depatuxizumab, depatuxizumab mafodotin (ABT-414), DFP-17729, dositinib, DS-2087, DZD-9008, E01001, E-JOC, epertinib, epitinib (HMPL-813), erlotinib, ES-072, FCN-411, FHND-9041, furmonertinib, FWD-1509, GB-263, GC-1118A, gefitinib, GMA-204, GR-1401, Hemay-022, HLX-07, HS-627, I-010, icotinib, imgatuzumab, IN-A008, JMT-101, JRF-103, JS-111, JS-113, JZB-28, KN-023, KN-026, KP-673, lapatinib, larotinib, lazertinib, LL-191, LYN 205, M1231, maihuatinib, marizomib, mobocertinib, MP-0274, MRG003, naputinib tosilate, nazartinib, necitumumab, neptinib, nimotuzumab, NRC-2694-A, NT-004, OBX1-012, olafertinib, olmutinib, ORIC-114, oritinib, osimertinib, panitumumab, pirotinib, poziotinib, PRB-001, pyrotinib, QL-1203, SCT-200, serclutamab, SHR-A1307, SIM-200, SPH-1188, SSGJ-612, SYN-004, TAD-011, tarloxotinib, TAS-6417, TGRX-360, theliatinib (HMPL-309), TPC-064, TQB-3804, TY-9591, WJ-13404, WSD-0922, XZP-5809, yinlitinib maleate, YK-029A, YZJ-0318, zorifertinib, and ZSP-0391.

In some embodiments of a method disclosed herein, the EGFR inhibitor is erlotinib.

In some embodiments of a method disclosed herein, the erlotinib is administered to the subject at a dose of 150 mg PO daily or 100 mg PO daily or 50 mg PO daily.

In some embodiments of a method disclosed herein, the dose of Compound 1 is between about 20 to about 40 mg, about 40 to about 80 mg, about 80 to about 120 mg, about 120 mg to about 160 mg, or about 160 mg to about 200 mg.

In some embodiments of a method disclosed herein, the cancer is colorectal cancer, esophageal cancer, gastric cancer, gastroesophageal junction (GEJ) cancer, head and neck squamous cell carcinoma, non-small cell lung cancer, or subtype squamous cell carcinoma.

In some embodiments of a method disclosed herein, the oncogene amplification comprises an amplification of FGFR1, FGFR2, FGFR3 or a combination thereof and wherein the treatment further comprises administering an FGFR inhibitor.

In some embodiments of a method disclosed herein, the application of FGFR1, FGFR2, FGFR3 or FGFR4 is an amplification of a wildtype FGFR1, FGFR2, FGFR3 or FGFR4.

In some embodiments of a method disclosed herein, the FGFR inhibitor is selected from the group consisting of 3D-185, ABSK-011, ABSK-012, ABSK-061, ABSK-091, aldafermin, alofanib, AST-56100, AZD-4547, bemarituzumab, BFKB-8488A, BGS-2219, BIO-1262, BPI-17509, BPI-43487, CPL-304-110, derazantinib, E-7090, erdafitinib, EVER-4010001, EVT-601, FGF-401, fisogatinib, FPI-1966, futibatinib, gunagratinib, H3B-6527, HH-185, HMPL-453, HS-236, ICP-105, ICP-192, infigratinib, JAB-6000, KIN-3248, M-6123, MAX-40279, OM-RCA-001, pemigatinib, RLY-4008, rogaratinib, SAR-439115, SAR-442501, SC-0011, SY-4798, TT-00434, zoligratinib (FF-284), and WXSH-0011.

In some embodiments of a method disclosed herein, the FGFR inhibitor is pemigatinib.

In some embodiments of a method disclosed herein, the pemigatinib is administered to the subject at a dose of 13.5 mg PO daily or 9 PO mg or 4.5 mg PO daily and the dose is administered once daily for 14 days followed by 7 sequential days without administration of pemigatinib.

In some embodiments of a method disclosed herein, the dose of Compound 1 is between about 20 to about 40 mg, about 40 to about 80 mg, about 80 to about 120 mg, about 120 mg to about 160 mg, or about 160 mg to about 200 mg.

In some embodiments of a method disclosed herein, the FGFR inhibitor is futibatinib.

In some embodiments of a method disclosed herein, the futibatinib is administered to the subject at a dose of 20 mg PO daily.

In some embodiments of a method disclosed herein, the dose of Compound 1 is between about 20 to about 40 mg, about 40 to about 80 mg, about 80 to about 120 mg, about 120 mg to about 160 mg, or about 160 mg to about 200 mg.

In some embodiments of a method disclosed herein, the cancer is breast cancer, cholangiocarcinoma, esophageal cancer, head and neck squamous cell carcinoma, non-small cell lung cancer, stomach cancer, or subtype squamous cell carcinoma.

In some embodiments of a method disclosed herein, the oncogene amplification comprises an amplification of CDK4, CDK6 or a combination thereof and wherein the treatment further comprises administering a CDK4/6 inhibitor.

In some embodiments of a method disclosed herein, the CDK4/6 inhibitor is selected from the group consisting of abemaciclib, AG-122275, AM-5992, AT-7519, AU2-94, auceliciclib, BEBT-209, BPI-1178, BPI-16350, CS-3002, fascaplysin, FCN-437, FN-1501, GLR-2007, GW-491619, HEC-80797, HS-10342, IIIM-290, IIIM-985, lerociclib, milciclib maleate, MM-D37K, MS-140, NP-102, NUV-422, ON-123300, palbociclib, PF-06842874, PF-06873600, PF-07220060, QHRD-110, R-547, RGB-286199, RGT-419B, ribociclib, riviciclib, RO-0505124, SHR-6390, THR-53, THR-79, TQB-3303, TQB-3616, trilaciclib, TY-302, TY-302, voruciclib, VS2-370, WXWH-0240, XH-30002, and XZP-3287.

In some embodiments of a method disclosed herein, the CDK4/6 inhibitor is abemaciclib.

In some embodiments of a method disclosed herein, the abemaciclib is administered to the subject at a dose of about 50 mg twice daily, about 100 mg twice daily, or 150 mg twice daily.

In some embodiments of a method disclosed herein, the dose of Compound 1 is between about 10 mg to about 20 mg, about 20 mg to about 40 mg, about 40 to about 80 mg, or about 80 to about 120 mg.

In some embodiments of a method disclosed herein, the cancer is esophageal cancer, non-small cell lung cancer, a sarcoma, or stomach cancer.

In some embodiments of a method disclosed herein, the therapeutic response comprises a reduction in the level of oncogene amplification in the tumor or tumor cells after treatment as compared to the level of oncogene amplification in the tumor or tumor cells prior to treatment.

In some embodiments of a method disclosed herein, the method further comprises obtaining a diagnostic indicator of oncogene amplification in a biological sample from the subject.

In some embodiments of a method disclosed herein, the diagnostic indicator is obtained prior to a first administration of Compound 1.

In some embodiments of a method disclosed herein, the diagnostic indicator is obtained subsequent to a first administration of Compound 1.

In some embodiments of a method disclosed herein, the diagnostic indicator is obtained subsequent to multiple administrations of Compound 1.

In some embodiments of a method disclosed herein, the diagnostic indicator results from a next generation sequencing (NGS)-based assay.

In some embodiments of a method disclosed herein, the diagnostic indicator results from a fluorescence in situ hybridization (FISH) assay.

In some embodiments of a method disclosed herein, the diagnostic indicator comprises an indicator for ecDNA-derived oncogene amplification.

In some embodiments of a method disclosed herein, the diagnostic indicator is obtained from tumor or liquid biopsy.

In some embodiments of a method disclosed herein, the method further comprises assessing a sample from a subject for the presence or level of one or more of a gene amplification, a focal gene amplification, ecDNA, HSR, or an ecDNA signature.

In some embodiments of a method disclosed herein, the method further comprises obtaining information of the presence or level of one or more of a gene amplification, a focal gene amplification, ecDNA, HSR, or an ecDNA signature in the tumor or tumor cells from the subject prior to, during or subsequent to the administration of Compound 1.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows Compound 1 demonstrating anti-tumor activity as a single agent and in combination with erlotinib in the ecDNA+ EGFR amplified LU1206 PDX tumor model.

FIG. 2A shows Compound 1 demonstrating anti-tumor and PD activity as a single agent and in combination with infigratinib in the ecDNA+ FGFR2 amplified SNU-16 CDX tumor model (tumor volume).

FIG. 2B shows Compound 1 demonstrating anti-tumor and PD activity as a single agent and in combination with infigratinib in the ecDNA+ FGFR2 amplified SNU-16 CDX tumor model (gene copy number).

FIG. 2C shows Compound 1 demonstrating anti-tumor and PD activity as a single agent and in combination with infigratinib in the ecDNA+ FGFR2 amplified SNU-16 CDX tumor model (tumor volume).

FIG. 3A shows Compound 1 demonstrating anti-tumor activity as a single agent and in combination with palbociclib in the ecDNA+ CDK4 amplified SJSA-1 CDX tumor model.

FIG. 3B shows Compound 1 demonstrating anti-tumor activity as a single agent and in combination with palbociclib in the ecDNA+ CDK4 amplified SJSA-1 CDX tumor model.

FIG. 4A shows the tumor volume after treatment with Compound 1 (5 mg/kg PO QD×28D and 10 mg/kg PO QD×28D) and prexasertib (15 mg/kg SC Q14D (2 doses)) in the cell line derived xenograft (CDX) Kelly representing MYCN amplified neuroblastoma.

FIG. 4B shows the tumor volume after treatment with Compound 1 (50 mg/kg PO Q2D 2 wk ON/1 wk OFF/2 wk ON and 50 mg/kg PO×35D) and prexasertib (15 mg/kg SC Q14D (2 doses)) in the cell line derived xenograft (CDX) Kelly representing MYCN amplified neuroblastoma.

FIG. 4C shows the tumor volume after treatment with Compound 1 (100 mg/kg PO Q7D (4 doses) and 200 mg/kg PO Q7D (4 doses)) and prexasertib (15 mg/kg SC Q14D (2 doses)) in the cell line derived xenograft (CDX) Kelly representing MYCN amplified neuroblastoma.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “biological sample,” as used herein, generally refers to a sample derived from or obtained from a subject, such as a mammal (e.g., a human). Biological samples are contemplated to include but are not limited to, hair, fingernails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject.

The term “tumor” or “tumor cells” as used herein, generally refers to cells that grow and divide more than they should or do not die when they should. In some cases, tumor cells are present in a solid mass, such as a solid tumor, or in some cases, tumor cells are found in a non-solid form, such as in blood cancers. Tumor or tumor cells also can include metastasis or metastasizing cells, where cancer cells break away from the original (primary) tumor and may form a new tumor in other organs or tissues of the body.

The term “ecDNA signature” as used herein, generally refers to one or more characteristics common to tumors or tumor cells that are ecDNA+. In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, the ecDNA signature can include an increase in copy number (gene amplification) in conjunction with particular structural variations. In some cases, the ecDNA signature can include a focal amplification. In some cases, ecDNA signature includes a detection or identification of ecDNA using an imaging technology. In some cases, ecDNA signature does not include any imaging or direct detection of ecDNA.

### Compounds

Described herein are method of treating cancer in a subject in need thereof comprising administering to the subject a Chk1 inhibitor.

### Compound 1

In some embodiments, the Chk1 inhibitor is Compound 1, or a pharmaceutically acceptable salt thereof. Compound 1 is 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

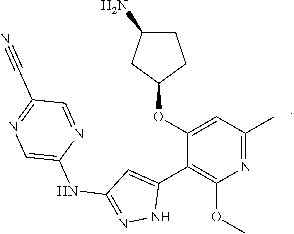 [SMILES: COc1nc(C)cc(O[C@@H]2CC[C@H](N)C2)c1-c1cc(Nc2cnc(C#N)cn2)n[nH]1]

In some embodiments, Compound 1 is a free base.

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, tautomer, or stereoisomer thereof, include isotopes of hydrogen, carbon, nitrogen, oxygen, phosphorous, sulfur, fluorine, and chloride, such as ²H, ³H, ³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof that contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, tautomer, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein that comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like. Representative salts include the alkali or alkaline earth salts, like lithium, sodium, potassium, calcium, and magnesium, and aluminum salts and the like of the tetrazole.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature.

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts,” Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, tautomer, or stereoisomer thereof, and a pharmaceutically acceptable excipient.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as increased overall response rate, increased duration of response, more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, epidural, or intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached.

### Methods

Disclosed herein is a method of treating cancer in a subject in need thereof comprising administering to the subject a pharmaceutical composition comprising Compound 1, or a pharmaceutically acceptable salt thereof. In some embodiments, the Chk1 inhibitor is Compound 1, or a pharmaceutically acceptable salt thereof.

In some embodiments of a method disclosed herein, the subject experiences a therapeutic response.

In some embodiments of a method disclosed herein, the therapeutic response comprises a reduction in the level of oncogene amplification in the tumor or tumor cells after treatment as compared to the level of oncogene amplification in the tumor or tumor cells prior to treatment.

In some embodiments of a method disclosed herein, the therapeutic response comprises reduction in of one or more of tumor growth, tumor size, number of tumor cells, or tumor metastasis as compared to prior to treatment.

In some embodiments of a method disclosed herein, the therapeutic response comprises a therapeutic benefit. In some embodiments of a method disclosed herein, the therapeutic benefit is stable disease (SD). In some embodiments of a method disclosed herein, the therapeutic benefit is partial response (PR). In some embodiments of a method disclosed herein, the therapeutic benefit is complete response. In some embodiments of a method disclosed herein, a complete response is determined by RECISTv1.1 (or RANO for GBM). In some embodiments of a method disclosed herein, the therapeutic benefit is progression-free survival. In some embodiments of a method disclosed herein, the therapeutic benefit is overall survival.

In some embodiments of a method disclosed herein, the cancer comprises a solid tumor.

In some embodiments of a method disclosed herein, the cancer comprises a locally advanced or metastatic non-resectable solid tumor.

In some embodiments of a method disclosed herein, the cancer comprises a tumor or tumor cells harboring an oncogene amplification.

In some embodiments of a method disclosed herein, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments of a method disclosed herein, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FGFR4, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments of a method disclosed herein, the oncogene amplification comprises an amplification of FGFR1, FGFR2, FGFR3, or a combination thereof.

In some embodiments of a method disclosed herein, the oncogene amplification comprises an amplification of FGFR1, FGFR2, FGFR3, FGFR4, or a combination thereof.

In some embodiments of a method disclosed herein, the oncogene amplification comprises an amplification of FGFR1, FGFR2, FGFR3, or a combination thereof and wherein the treatment further comprises administering an FGFR inhibitor.

In some embodiments of a method disclosed herein, the oncogene amplification comprises an amplification of FGFR1, FGFR2, FGFR3, FGFR4, or a combination thereof and wherein the treatment further comprises administering an FGFR inhibitor.

In some embodiments of a method disclosed herein, the oncogene amplification comprises CDK4, CDK6, EGFR, FGFR1, FGFR2, or FGFR3.

In some embodiments of a method disclosed herein, the oncogene amplification comprises CDK4, CDK6, EGFR, FGFR1, FGFR2, FGFR3, or FGFR4.

In some embodiments of a method disclosed herein, the oncogene amplification comprises an amplification of EGFR. In some embodiments of a method disclosed herein, the oncogene amplification comprises an amplification of EGFR and wherein the treatment further comprises administering an EGFR inhibitor.

In some embodiments of a method disclosed herein, the oncogene amplification comprises an amplification of CDK4, CDK6, or a combination thereof. In some embodiments of a method disclosed herein, the oncogene amplification comprises an amplification of CDK4, CDK6, or a combination thereof and wherein the treatment further comprises administering a CDK4/6 inhibitor.

In some embodiments of a method disclosed herein, the oncogene amplification resides on ecDNA.

In some embodiments of a method disclosed herein, the oncogene amplification resides on one or more chromosomal loci.

In some embodiments of a method disclosed herein, the oncogene amplification is an ecDNA-derived amplification.

In some embodiments of a method disclosed herein, the oncogene amplification has a copy number of at least 6, at least 8, at least 10, at least 15, at least 20 or more than 20 copies of the oncogene or portion thereof.

In some embodiments of a method disclosed herein, the cancer includes malignant tumors whose size can be decreased, whose growth or spread can be slowed or halted, or whose symptom is in remission or alleviated, reduced, and/or completely cured by deleting or suppressing and/or inhibiting functions of Chk1. Malignant tumors of interest are, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, squamous cell lung carcinoma, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors (e.g., soft tissue sarcomas and osteosarcomas), skin cancer, brain tumor (e.g., glioblastoma) and the like.

In some embodiments of a method disclosed herein, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include acute myeloid leukemia, adrenal cortical cancer, adrenal gland cancer, bladder cancer, bone cancer, brain cancer, breast cancer (e.g., ductal carcinoma, lobular carcinoma, primary, metastatic), breast cancer, cancer of the endocrine system, cancer of the hepatic stellate cells, cancer of the pancreatic stellate cells, cervical cancer, colon cancer, colorectal cancer, ductal carcinoma, endometrial cancer, esophageal cancer, gastric cancer, genitourinary tract cancer, glioblastoma, glioma, head and neck cancer, hepatocellular carcinoma, Hodgkin's Disease, kidney cancer, leukemia (e.g., lymphoblastic leukemia, chronic lymphocytic leukemia, hairy cell leukemia), liver cancer (e.g., hepatocellular carcinoma), lobular carcinoma, lung cancer (e.g., non-small cell lung carcinoma, squamous cell lung carcinoma, adenocarcinoma, large cell lung carcinoma, small cell lung carcinoma, carcinoid, sarcoma), lymph node cancer, lymphoma (e.g., Mantel cell lymphoma, follicular lymphoma, diffuse large B-cell lymphoma, marginal zona lymphoma, Burkitt's lymphoma, Non-Hodgkin's Lymphoma) malignant carcinoid, malignant hypercalcemia, malignant pancreatic insulinoma, medullary thyroid cancer, Medulloblastoma, melanoma, mesothelioma, multiple myeloma muscle cancer, neoplasms of the endocrine or exocrine pancreas, neuroblastoma, ovarian cancer, Paget's Disease of the Nipple, pancreatic cancer, papillary thyroid cancer, Phyllodes Tumors, premalignant skin lesions, primary thrombocytosis, prostate cancer (e.g. castration-resistant prostate cancer) rhabdomyosarcoma, salivary gland cancer, sarcoma, soft tissue sarcoma, squamous cell carcinoma (e.g., head, neck, or esophagus), stomach cancer, testicular cancer, thyroid cancer, urinary bladder cancer, or uterine cancer. In embodiments, the cancer is selected from bladder cancer, breast cancer, colon cancer, esophageal cancer, esophageal cancer, glioblastoma, liver cancer, lung cancer, ovarian cancer, pancreatic cancer, prostate cancer, salivary gland cancer, soft tissue sarcoma, squamous cell lung carcinoma, stomach cancer, and uterine cancer.

In some embodiments of a method disclosed herein, the cancer is an ovarian cancer. In some embodiments of a method disclosed herein, the ovarian cancer is a platinum resistant high-grade serous ovarian cancer, a primary peritoneal cancer, or a fallopian tube cancer.

In some embodiments of a method disclosed herein, the cancer is a uterine cancer. In some embodiments of a method disclosed herein, the uterine cancer is a high-grade endometrial carcinoma, a uterine serous carcinoma or a uterine carcinosarcoma.

In some embodiments of a method disclosed herein, the cancer is a neuroblastoma.

In some embodiments of a method disclosed herein, the cancer is colorectal cancer, esophageal cancer, gastric cancer, gastroesophageal junction (GEJ) cancer, head and neck squamous cell carcinoma, non-small cell lung cancer, or subtype squamous cell carcinoma.

In some embodiments of a method disclosed herein, the cancer is breast cancer or head, esophageal cancer, neck squamous cell carcinoma, non-small cell lung cancer, stomach cancer, or subtype squamous cell carcinoma.

In some embodiments of a method disclosed herein, the cancer is esophageal cancer, non-small cell lung cancer, a sarcoma, or stomach cancer. In some embodiments of a method disclosed herein, the cancer is esophageal cancer. In some embodiments of a method disclosed herein, the cancer is non-small cell lung cancer. In some embodiments of a method disclosed herein, the cancer is a sarcoma. In some embodiments of a method disclosed herein, the cancer is stomach cancer.

In some embodiments of a method disclosed herein, the subject has undergone one or more prior therapies.

In some embodiments of a method disclosed herein, the subject was non-responsive to the one or more prior therapies.

In some embodiments of a method disclosed herein, the subject developed resistance to the one or more prior therapies.

In some embodiments of a method disclosed herein, the one or more prior therapies is chemotherapies.

In some embodiments of a method disclosed herein, the one or more prior therapies is a PD1 antibody.

In some embodiments of a method disclosed herein, the one or more prior therapies is a PD-L1 antibody.

In some embodiments of a method disclosed herein, the one or more prior therapies is a CTLA4 checkpoint inhibitor.

In some embodiments of a method disclosed herein, the one or more prior therapies is VEGF targeting therapies (e.g., bevacizumab for ovarian cancer).

Disclosed herein is a method of treating cancer in a subject in need thereof comprising administering to the subject a pharmaceutical composition comprising Compound 1, or a pharmaceutically acceptable salt thereof, wherein the method further comprises obtaining a diagnostic indicator of oncogene amplification in a biological sample from the subject.

In some embodiments of a method disclosed herein, the diagnostic indicator is obtained prior to a first administration of Compound 1, or a pharmaceutically acceptable salt thereof.

In some embodiments of a method disclosed herein, the diagnostic indicator is obtained subsequent to a first administration of Compound 1, or a pharmaceutically acceptable salt thereof.

In some embodiments of a method disclosed herein, the diagnostic indicator is obtained subsequent to multiple administrations of Compound 1, or a pharmaceutically acceptable salt thereof.

In some embodiments of a method disclosed herein, the diagnostic indicator results from a next generation sequencing (NGS)-based assay.

In some embodiments of a method disclosed herein, the diagnostic indicator results from a fluorescence in situ hybridization (FISH) assay.

In some embodiments of a method disclosed herein, the diagnostic indicator comprises an indicator for ecDNA-derived oncogene amplification.

In some embodiments of a method disclosed herein, the diagnostic indicator is obtained from tumor or liquid biopsy.

In some embodiments of a method disclosed herein, the method further comprises assessing a sample from a subject for the presence or level of one or more of a gene amplification, a focal gene amplification, ecDNA, HSR, or an ecDNA signature.

In some embodiments of a method disclosed herein, the method further comprises obtaining information of the presence or level of one or more of a gene amplification, a focal gene amplification, ecDNA, HSR, or an ecDNA signature in the tumor or tumor cells from the subject prior to, during or subsequent to the administration of Compound 1, or a pharmaceutically acceptable salt thereof.

In some embodiments of a method disclosed herein, the cancer is an ovarian cancer. In some embodiments of a method disclosed herein, the ovarian cancer is a platinum resistant high-grade serous ovarian cancer, a primary peritoneal cancer, or a fallopian tube cancer.

In some embodiments of a method disclosed herein, the cancer is a uterine cancer. In some embodiments of a method disclosed herein, the uterine cancer is a high-grade endometrial carcinoma, a uterine serous carcinoma or a uterine carcinosarcoma.

In some embodiments of a method disclosed herein, the cancer is a neuroblastoma.

In some embodiments of a method disclosed herein, the cancer is colorectal cancer, esophageal cancer, gastric cancer, gastroesophageal junction (GEJ) cancer, head and neck squamous cell carcinoma, non-small cell lung cancer, or subtype squamous cell carcinoma.

In some embodiments of a method disclosed herein, the cancer is breast cancer, cholangiocarcinoma, esophageal cancer, head and neck squamous cell carcinoma, non-small cell lung cancer, stomach cancer, or subtype squamous cell carcinoma.

In some embodiments of a method disclosed herein, the cancer is esophageal cancer, non-small cell lung cancer, a sarcoma, or stomach cancer.

In some embodiments of a method disclosed herein, the cancer is bladder cancer, breast cancer, esophageal cancer, gastric cancer, glioblastoma, head and neck squamous cell carcinoma, liposarcoma, non-small cell lung adenocarcinoma, non-small cell lung cancer, non-small cell squamous lung cancer, or ovarian cancer.

In some embodiments of a method disclosed herein, the cancer is bladder cancer, breast cancer, endometrial cancer, esophageal cancer, gastric cancer, head and neck squamous cell carcinoma, liposarcoma, non-small cell lung adenocarcinoma, non-small cell lung cancer, non-small cell squamous lung cancer, or ovarian cancer.

### Oncogene Amplification

Oncogene amplification-associated tumors are a segment of the cancer population with an extremely high unmet need. Patients whose cancers harbor high-copy oncogene amplification have significantly worse survival compared with the broader cancer population. Pan-cancer analysis of oncogene-amplified tumors, cross-referenced with the Surveillance, Epidemiology, and End Results program data, indicates that, in the US alone, this population represents more than 400,000 newly diagnosed cancer patients each year across multiple tumor types.

Despite the enormous health benefits and improvements in survival afforded by precision medicine and targeted therapies for cancer, these therapies have unfortunately proven largely non-efficacious in the oncogene amplification population. In addition, immune checkpoint inhibitors (e.g., pembrolizumab) might perform poorly in oncogene-amplified cancer populations and hyper-progression has been associated with oncogene-amplified tumor settings.

To date, HER2 inhibitors (e.g., trastuzumab) for HER2-overexpressing breast cancer, gastroesophageal junction cancer, and gastric cancer are the only targeted therapies approved in oncogene amplified (or overexpressed) cancer populations, with breast cancer being the only single agent approval. Targeted agents that have shown efficacy in patients whose cancers are driven by oncogene point mutations, gene fusions or skipping deletions have generally failed to demonstrate robust efficacy in patients whose tumors are driven by oncogene amplification. This lack of approved therapies is despite extensive clinical testing of targeted agents in oncogene-amplified cancer populations including EGFR inhibitors in EGFR-amplified glioblastoma multiforme, FGFR inhibitors in FGFR-amplified cancers, and CDK4/6 inhibitors in CDK4-amplified liposarcoma. These clinical data have resulted in the misconception that oncogene amplification may not be a relevant cancer driver. This erroneous conclusion is despite extensive data to the contrary. Recurrent focal copy number amplification and overexpression of established oncogene drivers (otherwise activated by mutation and/or gene fusion), as well as antitumor efficacy of targeted inhibition (genetic and pharmacologic) in short-term preclinical cancer models establishes that amplifications are drivers, even if targeted therapy treatment approaches are generally not translating to prolonged clinical benefit. However, the above disconnect suggests that cancers driven by oncogene amplifications are biologically different from other tumors and require a new therapeutic paradigm. Accordingly, improved understanding of oncogene amplification biology is necessary, with the objectives of advancing new therapeutic approaches, pharmaceutical targets, and new molecular entities for this high unmet need patient population.

### Role of Extrachromosomal DNA in Oncogene Amplification

Chromosomal instability and tumor heterogeneity have been suggested to account for many targeted therapy failures. Consistent with this hypothesis, oncogene amplification is a consequence of prior or ongoing chromosomal instability arising through either numerical and/or structural alterations in chromosomes and can give rise to ecDNA. It has long been recognized that oncogenes can be amplified not only on chromosomes but also on ecDNA, originally referred to as “double minutes.” However, the frequency, importance, and specific roles of ecDNA in cancer biology have not been well understood until recently.

Some of the most prevalent driver oncogenes are encoded on ecDNA or ecDNA-derived amplifications and can confer a selective advantage to cancer cells. These oncogenes amplified on ecDNA (or ecDNA-derived DNA) have several features that distinguish them from chromosomally localized oncogene amplifications:
1. ecDNA lack centromeres, thus, in contrast to chromosomally localized amplification states, they segregate unequally into daughter cells during cell division. This property supports a non-Mendelian inheritance pattern, enabling extreme gene copy number changes in relatively few cell divisions and leads to extensive copy number heterogeneity driving adaptability and tumor evolution.
2. ecDNA are epigenetically dysregulated and contain accessible chromatin and hyper-transcribed gene regions that are often more actively expressed than chromosomally located genes. Focal amplifications, i.e., focal high copy number oncogene amplification frequently occur on extrachromosomal DNA (ecDNA). ecDNA oncogene amplifications (i.e. ecDNA-derived focal amplifications) can exist dynamically in different states, extrachromosomal or reintegrated into a chromosome. In some cases, the focal amplification is an ecDNA-derived amplification and the amplification has reintegrated into a chromosome. In some cases, the focal amplification is on ecDNA (i.e., extrachromosomal location).

These features distinguish ecDNA and ecDNA-derived focal amplifications from other forms of oncogene amplification and facilitate a level of genomic plasticity and adaptability beyond chromosomal amplification that enable tumors to evade environmental insults, including targeted therapeutic pressure. New therapeutic approaches that interfere with ecDNA function are necessary to overcome ecDNA mediated adaptation in oncogene-amplified tumors.

ecDNA-enabled (e.g., ecDNA and ecDNA-derived) oncogene amplifications are a primary driver of oncogenesis, play a critical role in driving tumor heterogeneity, and enable cancer cells to rapidly become resistant to targeted oncogene therapies. ecDNA-enabled oncogene amplifications were observed in nearly half of all human cancer types but almost never found in normal cell. ecDNA-enabled oncogene amplifications can be found in approximately 14% of primary cancer specimens and that more than half of all high-copy number oncogene amplifications (i.e., copy number value >8) reside on ecDNA. Further, many of the most aggressive tumor types contain the highest prevalence of ecDNA, including approximately 60% of glioblastoma multiforme and just under 50% of sarcomas.

Patients whose cancers harbor ecDNA experience significantly shorter survival than cancer patients whose tumors are driven by other molecular lesions, even when controlled for tumor type. These data strongly indicate that patients with ecDNA-enabled cancers require a new therapeutic paradigm to address this large unmet need.

### Role of Extrachromosomal DNA in Therapeutic Resistance

The unique features of ecDNA, coupled with the remarkable genome plasticity of ecDNA-enabled tumors, contribute to the tumor's aggressive nature and ability to evade therapeutic pressure via rapid genomic evolution. The first demonstration of therapeutic resistance driven by ecDNA was in a mouse cancer cell line whereby methotrexate treatment led to high amplification of dihydrofolate reductase (DHFR) on ecDNA, and which was lost upon removal of methotrexate. Similar instances of DHFR ecDNA amplification have been recapitulated in multiple human cancer cell lines. Furthermore, amplification of drug efflux pump genes on ecDNA, including the family of ABC transporters, has been observed to mediate resistance to various chemotherapies. An equivalent role for ecDNA in providing resistance to more current targeted therapies has also been well established. Evasion of therapeutic response to the EGFR inhibitor erlotinib is facilitated by rapid loss of the population of EGFRvIII amplifications on ecDNA in patient-derived glioblastoma multiforme cells, contemporaneous with occurrence of a new cell population containing MDM2 amplification on ecDNA; this observed effect is consistent with an equivalent lack of response to EGFR inhibitors in patients. Preclinical studies in a gastric cancer cell line containing FGFR2 amplified on ecDNA demonstrated that cellular resistance to the pan-FGFR inhibitor infigratinib could be driven by oncogene dependency switching from FGFR2 amplification on ecDNA to a new, rapid amplification of EGFR on ecDNA. Strikingly, this dependency was reversed back to FGFR2 amplification on ecDNA under EGFR inhibitory pressure via erlotinib. In each case, the initial cell population was sensitive to the respective targeted therapy, resulting in short lived anti-proliferative effects lasting several weeks. Resistance and regrowth to the targeted therapies occurred coincident with switching of the amplified oncogenes on ecDNA. The rapid rate of amplification change is unique to ecDNA and helps account for the intrinsic targeted therapy resistance of de novo oncogene-amplified cancers.

Similarly, mutant oncogenes (e.g., BRAFV600E and KRASG12C) can be amplified on ecDNA as a resistance mechanism to corresponding targeted therapies (e.g., BRAF/MEK or KRAS inhibitors). For example, a mutant BRAFV600E melanoma cell line developed ecDNA-enabled amplification of BRAFV600E after exposure to BRAF/MEK dual inhibition. This phenomenon has also been documented in clinical cases. Relatedly, numerous putative acquired resistance mechanisms to the KRASG12C inhibitor adagrasib have been reported and of these, a high-level focal amplification of KRASG12C on ecDNA, confirmed in vitro and in vivo, conferred resistance to both clinically validated KRASG12C inhibitors, adagrasib and sotorasib.

Collectively, these studies highlight the striking genomic plasticity and precipitous rise of ecDNA enabled oncogene amplification that enables cancer cells to adapt rapidly to therapeutic pressure. In conclusion, cancers driven by ecDNA-enabled oncogene amplification are biologically different from other oncogene activated tumors and require a new therapeutic paradigm.

### Dosing/Administration

Disclosed herein is a method of treating cancer in a subject in need thereof comprising administering to the subject a pharmaceutical composition comprising Compound 1, or a pharmaceutically acceptable salt thereof.

### Rationale for Human Starting Dose of Compound 1

GLP-compliant repeat dose toxicology studies were conducted in rats and dogs following oral gavage Q2D with exposure duration of up to 29 days. The main effects observed in both species were related to bone marrow suppression/depletion and gastrointestinal toxicity, and were considered an on-target pharmacological effect of Compound 1 that is directly associated with CHK1 inhibition. Partial or complete reversibility of Compound 1-related major changes was demonstrated in all tissues, and there was no major unexpected toxicity identified.

In some embodiments of a method disclosed herein, Compound 1 is administered at a dose of about 10 mg to about 800 mg.

In some embodiments of a method disclosed herein, Compound 1 is administered at a dose of about 10 mg to about 400 mg.

In some embodiments of a method disclosed herein, Compound 1 is administered at a dose of about 10 mg to about 400 mg, whereby the subject experiences a therapeutic response.

In some embodiments of a method disclosed herein, the dose of Compound 1 is between about 10 mg to about 20 mg, about 20 mg to about 40 mg, about 40 mg to about 80 mg, about 80 mg to about 120 mg, about 120 mg to about 160 mg, about 160 mg to about 200 mg, or about 200 mg to about 400 mg.

In some embodiments of a method disclosed herein, the dose of Compound 1 is between about 10 mg to about 20 mg, about 20 mg to about 40 mg, about 40 mg to about 80 mg, about 80 mg to about 120 mg, about 120 mg to about 160 mg, about 160 mg to about 200 mg, about 200 mg to about 240 mg, about 200 mg to about 400 mg.

In some embodiments of a method disclosed herein, the dose of Compound 1 is between about 20 to about 40 mg, about 40 to about 80 mg, about 80 to about 120 mg, about 120 mg to about 160 mg, or about 160 mg to about 200 mg.

In some embodiments of a method disclosed herein, the dose of Compound 1 is between about 20 to about 40 mg, about 40 to about 80 mg, about 80 to about 120 mg, about 120 mg to about 160 mg, about 160 mg to about 200 mg, or about 200 mg to about 240 mg.

In some embodiments of a method disclosed herein, the dose of Compound 1 is between about 10 mg to about 20 mg, about 20 mg to about 40 mg, about 40 to about 80 mg, or about 80 to about 120 mg.

In some embodiments of a method disclosed herein, the dose of Compound 1 is between about 80 to about 120 mg, about 120 mg to about 160 mg, about 160 mg to about 200 mg, or about 200 mg to about 240 mg.

In some embodiments of a method disclosed herein, the dose of Compound 1 is between about 10 mg to about 20 mg. In some embodiments of a method disclosed herein, the dose of Compound 1 is between about 20 mg to about 40 mg. In some embodiments of a method disclosed herein, the dose of Compound 1 is between about 40 mg to about 80 mg. In some embodiments of a method disclosed herein, the dose of Compound 1 is between about 80 mg to about 120 mg. In some embodiments of a method disclosed herein, the dose of Compound 1 is between about 120 mg to about 160 mg. In some embodiments of a method disclosed herein, the dose of Compound 1 is between about 160 mg to about 200 mg. In some embodiments of a method disclosed herein, the dose of Compound 1 is between about 200 mg to about 240 mg. In some embodiments of a method disclosed herein, the dose of Compound 1 is between about 200 mg to about 400 mg.

In some embodiments of a method disclosed herein, the dose of Compound 1 is about 10 mg, about 20 mg, about 40 mg, about 80 mg, about 120 mg, about 160 mg, or about 200 mg. In some embodiments of a method disclosed herein, the dose of Compound 1 is about 80 mg, about 120 mg, about 160 mg, about 200 mg or about 240 mg. In some embodiments of a method disclosed herein, the dose of Compound 1 is about 10 mg. In some embodiments of a method disclosed herein, the dose of Compound 1 is about 15 mg. In some embodiments of a method disclosed herein, the dose of Compound 1 is about 20 mg. In some embodiments of a method disclosed herein, the dose of Compound 1 is about 25 mg. In some embodiments of a method disclosed herein, the dose of Compound 1 is about 30 mg. In some embodiments of a method disclosed herein, the dose of Compound 1 is about 35 mg. In some embodiments of a method disclosed herein, the dose of Compound 1 is about 40 mg. In some embodiments of a method disclosed herein, the dose of Compound 1 is about 45 mg. In some embodiments of a method disclosed herein, the dose of Compound 1 is about 50 mg. In some embodiments of a method disclosed herein, the dose of Compound 1 is about 55 mg. In some embodiments of a method disclosed herein, the dose of Compound 1 is about 60 mg. In some embodiments of a method disclosed herein, the dose of Compound 1 is about 65 mg. In some embodiments of a method disclosed herein, the dose of Compound 1 is about 70 mg. In some embodiments of a method disclosed herein, the dose of Compound 1 is about 75 mg. In some embodiments of a method disclosed herein, the dose of Compound 1 is about 80 mg. In some embodiments of a method disclosed herein, the dose of Compound 1 is about 85 mg. In some embodiments of a method disclosed herein, the dose of Compound 1 is about 90 mg. In some embodiments of a method disclosed herein, the dose of Compound 1 is about 95 mg. In some embodiments of a method disclosed herein, the dose of Compound 1 is about 100 mg. In some embodiments of a method disclosed herein, the dose of Compound 1 is about 105 mg. In some embodiments of a method disclosed herein, the dose of Compound 1 is about 110 mg. In some embodiments of a method disclosed herein, the dose of Compound 1 is about 115 mg. In some embodiments of a method disclosed herein, the dose of Compound 1 is about 120 mg. In some embodiments of a method disclosed herein, the dose of Compound 1 is about 125 mg. In some embodiments of a method disclosed herein, the dose of Compound 1 is about 130 mg. In some embodiments of a method disclosed herein, the dose of Compound 1 is about 135 mg. In some embodiments of a method disclosed herein, the dose of Compound 1 is about 140 mg. In some embodiments of a method disclosed herein, the dose of Compound 1 is about 145 mg. In some embodiments of a method disclosed herein, the dose of Compound 1 is about 150 mg. In some embodiments of a method disclosed herein, the dose of Compound 1 is about 155 mg. In some embodiments of a method disclosed herein, the dose of Compound 1 is about 160 mg. In some embodiments of a method disclosed herein, the dose of Compound 1 is about 165 mg. In some embodiments of a method disclosed herein, the dose of Compound 1 is about 170 mg. In some embodiments of a method disclosed herein, the dose of Compound 1 is about 175 mg. In some embodiments of a method disclosed herein, the dose of Compound 1 is about 180 mg. In some embodiments of a method disclosed herein, the dose of Compound 1 is about 185 mg. In some embodiments of a method disclosed herein, the dose of Compound 1 is about 190 mg. In some embodiments of a method disclosed herein, the dose of Compound 1 is about 195 mg. In some embodiments of a method disclosed herein, the dose of Compound 1 is about 200 mg. In some embodiments of a method disclosed herein, the dose of Compound 1 is about 210 mg. In some embodiments of a method disclosed herein, the dose of Compound 1 is about 220 mg. In some embodiments of a method disclosed herein, the dose of Compound 1 is about 230 mg. In some embodiments of a method disclosed herein, the dose of Compound 1 is about 240 mg.

In some embodiments of a method disclosed herein, the composition is administered orally.

In some embodiments of a method disclosed herein, the composition is administered parentally.

In some embodiments of a method disclosed herein, the composition is administered every day.

In some embodiments of a method disclosed herein, the composition is administered every other day (Q2D).

In some embodiments of a method disclosed herein, the composition is administered on a cycle of day 1 and day 3 followed by a 4 day dosing holiday.

In some embodiments of a method disclosed herein, the composition is administered on a cycle of day 1 and day 2 followed by a 5 day dosing holiday.

In some embodiments of a method disclosed herein, the composition is administered on a cycle of day 1 followed by a 6 day dosing holiday (i.e. weekly).

In some embodiments of a method disclosed herein, the composition is administered on days 1, 2 and 3, followed by an 11 day dosing holiday.

In some embodiments of a method disclosed herein, the composition is administered every 3 days or weekly (QW).

In some embodiments of a method disclosed herein, the composition is administered every 3 days.

In some embodiments of a method disclosed herein, the composition is administered weekly (QW).

In some embodiments of a method disclosed herein, the composition is administered with a dosing holiday of 4 days, 4-7 days, 7 days, or 14 days.

In some embodiments of a method disclosed herein, the composition is administered with a dosing holiday of 4 days, 4-7 days, 5 days, 7 days, 11 days, or 14 days.

### Combination

Disclosed herein is a method of treating cancer in a subject in need thereof comprising administering to the subject a pharmaceutical composition comprising a Compound 1, or a pharmaceutically acceptable salt thereof and an additional therapeutic agent. In some embodiments, the Chk1 inhibitor is Compound 1, or a pharmaceutically acceptable salt thereof.

In some embodiments of a method disclosed herein, the additional therapeutic agent is an EGFR inhibitor.

In some embodiments of a method disclosed herein, the EGFR inhibitor is selected from the group consisting of 602, 705, 707, abivertinib, ABX-900, afatinib, agerafenib (RXDX-105), alflutinib mesylate, amivantamab, APL-1898, ASK-120067, aumolertinib (almonertinib), BBT-176, BDTX-1535, BDTX-189, BEBT-109, befortinib mesylate, beitatini, BLU-701, BLU-945, BPI-361175, BPI-7711, BPI-D0316, C-005, CDP1, cetuximab, CH-7233163, CK-101, CMAB-017, dacomitinib, depatuxizumab, depatuxizumab mafodotin (ABT-414), DFP-17729, dositinib, DS-2087, DZD-9008, E01001, E-10C, epertinib, epitinib (HMPL-813), erlotinib, ES-072, FCN-411, FHND-9041, furmonertinib, FWD-1509, GB-263, GC-1118A, gefitinib, GMA-204, GR-1401, Hemay-022, HLX-07, HS-627, I-010, icotinib, imgatuzumab, IN-A008, JMT-101, JRF-103, JS-111, JS-113, JZB-28, KN-023, KN-026, KP-673, lapatinib, larotinib, lazertinib, LL-191, LYN 205, M1231, maihuatinib, marizomib, mobocertinib, MP-0274, MRG003, naputinib tosilate, nazartinib, necitumumab, neptinib, nimotuzumab, NRC-2694-A, NT-004, OBX1-012, olafertinib, olmutinib, ORIC-114, oritinib, osimertinib, panitumumab, pirotinib, poziotinib, PRB-001, pyrotinib, QL-1203, SCT-200, serclutamab, SHR-A1307, SIM-200, SPH-1188, SSGJ-612, SYN-004, TAD-011, tarloxotinib, TAS-6417, TGRX-360, theliatinib (HMPL-309), TPC-064, TQB-3804, TY-9591, WJ-13404, WSD-0922, XZP-5809, yinlitinib maleate, YK-029A, YZJ-0318, zorifertinib, and ZSP-0391.

In some embodiments of a method disclosed herein, the EGFR inhibitor is erlotinib. Erlotinib (TARCEVA^{O}) is an oral small molecule inhibitor of the receptor tyrosine kinase EGFR. Early data showed anticancer activity in a several tumors including cancers of the lung and pancreas, and erlotinib was approved by the FDA in 2013 for EGFR mutant NSCLC with EGFR exon 19 deletions or exon 21 substitution mutations. Notably, erlotinib can inhibit wildtype EGFR and is not selective for mutant EGFR only.

The planned dosage of erlotinib is 150 mg orally once daily, given >1 hour before or 2 hours after food intake. This is the dose of erlotinib for treatment of NSCLC per the TARCEVA® United States Prescribing Information (USPI).

In some embodiments of a method disclosed herein, erlotinib is administered to the subject at a dose of 150 mg PO daily, 100 mg PO daily, or 50 mg PO daily.

In some embodiments of a method disclosed herein, the additional therapeutic agent is a FGFR inhibitor.

In some embodiments of a method disclosed herein, the FGFR inhibitor is selected from the group consisting of 3D-185, ABSK-011, ABSK-012, ABSK-061, ABSK-091, aldafermin, alofanib, AST-56100, AZD-4547, bemarituzumab, BFKB-8488A, BGS-2219, BIO-1262, BPI-17509, BPI-43487, CPL-304-110, derazantinib, E-7090, erdafitinib, EVER-4010001, EVT-601, FGF-401, fisogatinib, FPI-1966, futibatinib, gunagratinib, H3B-6527, HH-185, HMPL-453, HS-236, ICP-105, ICP-192, infigratinib, JAB-6000, KIN-3248, M-6123, MAX-40279, OM-RCA-001, pemigatinib, RLY-4008, rogaratinib, SAR-439115, SAR-442501, SC-0011, SY-4798, TT-00434, zoligratinib (FF-284), and WXSH-0011.

In some embodiments of a method disclosed herein, the FGFR inhibitor is pemigatinib. Pemigatinib (PEMAZYRE®) is an oral small molecule inhibitor of the receptor tyrosine kinase FGFR. Pemigatinib was first approved by the FDA in 2020 for previously treated, unresectable locally advanced or metastatic cholangiocarcinoma with a FGFR2 fusion or other rearrangement as detected by an FDA-approved test. Notably, pemigatinib can inhibit wildtype FGFR1, FGFR2, and FGFR3 receptors.

In some embodiments of a method disclosed herein, pemigatinib is administered to the subject at a dose of 13.5 mg PO daily, 9 PO mg, 4.5 mg PO daily and the dose is administered once daily for 14 days followed by 7 sequential days without administration of pemigatinib.

In some embodiments of a method disclosed herein, the FGFR inhibitor is futibatinib.

Futibatinib (LYTGOBI®) is indicated for the treatment of adults with previously treated, unresectable, locally advanced, or metastatic intrahepatic cholangiocarcinoma harboring fibroblast growth factor receptor 2 (FGFR2) gene fusions or other rearrangements. Futibatinib was approved for medical use in the United States in September 2022.

In some embodiments of a method disclosed herein, futibatinib is administered to the subject at a dose of 20 mg PO daily.

In some embodiments of a method disclosed herein, the additional therapeutic agent is a CDK4/6 inhibitor.

In some embodiments of a method disclosed herein, the CDK4/6 inhibitor is selected from the group consisting of abemaciclib, AG-122275, AM-5992, AT-7519, AU2-94, auceliciclib, BEBT-209, BPI-1178, BPI-16350, CS-3002, fascaplysin, FCN-437, FN-1501, GLR-2007, GW-491619, HEC-80797, HS-10342, IIIM-290, IIIM-985, lerociclib, milciclib maleate, MM-D37K, MS-140, NP-102, NUV-422, ON-123300, palbociclib, PF-06842874, PF-06873600, PF-07220060, QHRD-110, R-547, RGB-286199, RGT-419B, ribociclib, riviciclib, RO-0505124, SHR-6390, THR-53, THR-79, TQB-3303, TQB-3616, trilaciclib, TY-302, TY-302, voruciclib, VS2-370, WXWH-0240, XH-30002, and XZP-3287.

In some embodiments of a method disclosed herein, the CDK4/6 inhibitor is abemaciclib. Abemaciclib (VERZENIO®) is an oral small molecule inhibitor of CDK4/6. Abemaciclib was first approved by the FDA in 2017 for advanced or metastatic breast cancer, which is hormone receptor positive and HER-2 negative. Notably, abemaciclib can inhibit wildtype CDK4 and CDK6 receptors.

In some embodiments of a method disclosed herein, the abemaciclib is administered to the subject at a dose of about 50 mg twice daily, about 100 mg twice daily, or 150 mg twice daily.

In some embodiments of a method disclosed herein, the additional therapeutic agent is a BRAF inhibitor. In some embodiments of a method disclosed herein, the BRAF inhibitor is ABM-1310, agerafenib (RXDX-105), ARQ-736, ASN-003, AZ-304, AZ-628, BAL-3833, belvarafenib, BGB-3245, BI-882370, dabrafenib, DAY101, DP-2874, EBI-907, EBI-945, encorafenib, GDC-0879, lifirafenib, LUT-014, LYN 204, NMS-P285, NMS-P730, PF-04880594, PF-07284890, PLX-8394, RX-208, TL-241, UAI-201, UB-941, vemurafenib, VS-6766, or XL-281.

In some embodiments of a method disclosed herein, the additional therapeutic agent is a MDM2 or MDM4 inhibitor.

In some embodiments of a method disclosed herein, the MDM2 inhibitor is AD-021.32, ALRN-6924, APG-115, ASTX-295, ATSP-7041, BI-907828, CGM-097, CYC700, DS-5272, idasanutlin, KRT-232 (AMG-232), MD-224, MI-1061, MI-219, MI-43, MI-77301 (SAR405838, SAR299155), MK-8242, NU-8231, NVP-CGM097, OM-301, PXN-527, RAIN-32 (milademetan), RG7112 (R05045337), RG7388 (RG7775), Rigel-3, RO-2468, RO-5353, RO-5963, serdemetan (JNJ-26854165), SIL-43, siremadlin, or UBX-0101. In some embodiments of a method disclosed herein, the MDM4 inhibitor is17AAG, 489-PXN, ALRN-6924, APG-115, ATSP-7041, BI-907828, CTX1, FL-118, inulanolide A, K-178, or SAH-p53-8.

In some embodiments of a method disclosed herein, the additional therapeutic agent is a MET inhibitor.

In some embodiments of a method disclosed herein, the MET inhibitor is ABP-1130, BPI-1831, BPI-2021, BYON-3521, CG-203306, CX-1003, Debio-1144, EMD-94283, EMT-100, EMT-101, HE-003, LMV-12, LS-177, NX-125, OMO-2, PF-4254644, PRX-MET, PTX-2173, QBH-196, RP-1400, SAB-Y14, SAR-125844, SGX-126, SYD-3521, WXSH-0011, X-379, and XL-265, and anti-MET antibodies such as ABX-900, GB-263, FS-101, LY-3164530, LY-3343544, PMC-002, or SAIT-301. In some embodiments of a method disclosed herein, the MET inhibitor is ABN-401, ABT-700, AMG-208, AMG-337, ARGX-111, BAY-85-3474, BMS-817378, bozitinib, BPI-9016M, glumetinib, golvatinib tartrate, GST-HG161, HQP- 8361, 1-020, JNJ-38877605, kanitinib, merestinib, MK-2461, MK-8033, OMO-1, pamufetinib, S-49076, savolitinib, SPH-3348, tivantinib, SAR-125844, SCR-1515, and TPX-0022, or anti-MET antibodies such as APL-101, CKD-702, EMB-01, EMI-137, ficlatuzumab, HLX-55, HS-10241, MCLA-129, MT-8633, NOV-1105, RC-108, REGN-5093, SHR-A1403, Sym-015, or telisotuzumab vedotin. In some embodiments of a method disclosed herein, the MET inhibitor is amivantamab, capmatinib, crizotinib, or tepotinib.

In some embodiments of a method disclosed herein, the additional therapeutic agent is a KRAS inhibitor. In some embodiments of a method disclosed herein, the KRAS inhibitor is ABREV01, ARS-1620, APG-1842, ATG-012, BBP-454, BEPT-607, BI-2852, BI-1823911, BPI-421286, BTX-2541, COTI-219, IMM-1811900, JAB-21000, JAB-22000, JAB-23000, JAB-BX300, JP-002, KR-12, LYN 202, MRTX-1133, RAS-F, RMC-6236, RMC-6291, SDGR 5, STX-301, and YL-15293, or anti-KRAS antibodies such as SBT-100, SBT-102, or SBT-300. In some embodiments of a method disclosed herein, the KRAS inhibitor is adagrasib, ARS-3248, D-1553, GDC-6036, JDQ-443, LY3537982, sotorasib (AMG 510), or BI 1701963.

### EXAMPLES

### Example 1a—an Open-Label, Multicenter, First-In-Human, Dose-Escalation and Dose-Expansion, Phase 1/2 Study of Compound 1 and Compound 1 in Combination with Select Targeted Therapies in Subjects with Locally Advanced or Metastatic Solid Tumors with Oncogene Amplifications

### Objectives

### Primary Objectives

To assess the safety, tolerability, and DLTs of Compound 1 as a single agent administered orally at escalating dose levels in adult subjects with locally advanced or metastatic solid tumors with oncogene amplifications.

To assess the safety, tolerability, and DLTs of Compound 1 in combination with each of the following agents: an EGFR inhibitor (e.g., erlotinib), an FGFR inhibitor (e.g., pemigatinib or fitibatinib), or a CDK4/6 inhibitor (e.g., abemaciclib), at escalating dose levels in adults with locally advanced or metastatic solid tumors with corresponding oncogene amplifications.

To determine the maximum tolerated dose (MTD) and the recommended Phase 2 dose (RP2D) of Compound 1 as a single agent.

To determine the MTD and the RP2D of Compound 1 in combination with each of the following agents: an EGFR inhibitor (e.g., erlotinib), an FGFR inhibitor (e.g., pemigatinib or futibatinib), or a CDK4/6 inhibitor (e.g., abemaciclib).

### Secondary Objectives

To assess the PK of Compound 1 as a single agent and in combination with each of the following agents: an EGFR inhibitor (e.g., erlotinib), an FGFR inhibitor (e.g., pemigatinib or futibatinib), or a CDK4/6 inhibitor (e.g., abemaciclib).

To assess the preliminary antitumor activity of Compound 1 as a single agent and in combination with each of the following agents: an EGFR inhibitor (e.g., erlotinib), an FGFR inhibitor (e.g., pemigatinib or futibatinib), or a CDK4/6 inhibitor (e.g., abemaciclib).

### Exploratory Objectives

To assess predictive biomarkers for Compound 1 as a single agent and in combination with each of the following agents: an EGFR inhibitor (e.g., erlotinib), an FGFR inhibitor (e.g., pemigatinib or futibatinib), or a CDK4/6 inhibitor (e.g., abemaciclib) (e.g., ecDNA enabled oncogene amplification).

To assess pharmacodynamic biomarkers for Compound 1 as a single agent and in combination with each of the following agents: erlotinib, pemigatinib, or abemaciclib.

To assess the PK of each of the following agents: an EGFR inhibitor (e.g., erlotinib), an FGFR inhibitor (e.g., pemigatinib or futibatinib), or a CDK4/6 inhibitor (e.g., abemaciclib).

### Endpoints

### Primary Endpoints

TEAEs including determination of DLTs and SAEs. AEs will be assessed, and causality and severity will be assigned by using the National Cancer Institute Common Terminology Criteria for Adverse Events (NCI CTCAE), Version 5.0.

### Secondary Endpoints

PK parameters derived for Compound 1 as a single agent and in combination with each of an EGFR inhibitor (e.g., erlotinib), an FGFR inhibitor (e.g., pemigatinib or futibatinib), or a CDK4/6 inhibitor (e.g., abemaciclib):
Plasma PK parameters (maximum plasma concentration [Cₘₐₓ], trough concentration [C_{trough}], time to maximum plasma concentration [Tₘₐₓ] and area under the concentration-time curve from 0 to last quantifiable concentration [AUC₀₋ₗₐₛₜ] on Day 1 and Day 15) will be estimated using standard non-compartmental PK analysis.
Other PK parameters (terminal half-life [t_{1/2}], clearance, and accumulation ratios based on Cₘₐₓ, C_{trough}, and AUC [RCₘₐₓ, RC_{trough}, and RC_{AUC}, respectively]) will be evaluated if data permit.

Clinical response including objective response rate (ORR), duration of response (DOR), disease control rate (DCR), progression-free survival (PFS), and overall survival (OS) to Compound 1 or Compound 1 in combination with each of select targeted therapies (i.e., an EGFR inhibitor (e.g., erlotinib), an FGFR inhibitor (e.g., pemigatinib or futibatinib), or a CDK4/6 inhibitor (e.g., abemaciclib)), per RECIST v1.1.
ORR will be calculated as the sum of subjects with best overall response of complete response (CR) or partial response (PR).
DOR will be calculated as the time from earliest date of documented CR or PR until documented disease progression or death (by any cause, in the absence of progression). In progression-free subjects, DOR will be censored at the last evaluable tumor assessment following the earliest date of documented CR or PR.
DCR will be calculated as the sum of subjects with best overall response of CR, PR, or stable disease (SD).
PFS is defined as the time from start of study treatment, until documented disease progression or death (by any cause, in the absence of progression). In progression-free subjects, PFS will be censored at the last evaluable tumor assessment.
OS is defined as the time from start of study treatment, until death (by any cause) or date of censoring. Subjects alive or those lost to follow-up will be censored at the last date of contact (or last date known to be alive).

### Exploratory Endpoints

Potential predictive biomarkers for Compound 1 in tissue will be evaluated by NGS for cancer associated alterations and for ecDNA using ecDNA imaging technology; a subset of samples will be evaluated for oncogene amplification by fluorescent in situ hybridization (FISH). Plasma circulating tumor DNA (ctDNA) will be evaluated by NGS for oncogenic alterations and the presence of ecDNA.

### Overall Study Design

Compound 1 is an orally available, potent, and selective small molecule inhibitor of Chk1 intended as a tumor agnostic treatment for patients with locally advanced or metastatic solid tumors with oncogene amplification on ecDNA. This is a First in Human (FIH), open-label, non-randomized, 3-part, Phase 1/2 study to determine the safety profile and identify the MTD and RP2D of Compound 1 administered as a single agent or in combination with a select targeted therapy, i.e., an EGFR inhibitor (e.g., erlotinib), an FGFR inhibitor (e.g., pemigatinib or futibatinib), or a CDK4/6 inhibitor (e.g., abemaciclib). Compound 1 will be administered orally every other day to subjects with locally advanced or metastatic non-resectable solid tumors harboring oncogene amplifications, whose disease has progressed despite all standard therapies or for whom no further standard or clinically acceptable therapy exists.

Part 1 is a single agent Compound 1 dose escalation and dose expansion; Part 2 is a dose escalation of Compound 1 in combination with either an EGFR inhibitor (e.g., erlotinib), an FGFR inhibitor (e.g., pemigatinib or futibatinib), or a CDK4/6 inhibitor (e.g., abemaciclib); and Part 3 will be a dose expansion of Compound 1 in combination with either an EGFR inhibitor (e.g., erlotinib), an FGFR inhibitor (e.g., pemigatinib or futibatinib), or a CDK4/6 inhibitor (e.g., abemaciclib).

Subjects recruited for any part of the study will provide informed consent and will undergo Screening before study participation. After a Screening period of up to 28 days, qualified subjects will be enrolled to receive their assigned dose regimen of Compound 1 as a single agent or in combination with either an EGFR inhibitor (e.g., erlotinib), an FGFR inhibitor (e.g., pemigatinib or futibatinib), or a CDK4/6 inhibitor (e.g., abemaciclib). In all parts of the study, subjects will be treated until progression of disease, development of unacceptable toxicity, or withdrawal from the study or withdrawal of consent, whichever occurs first.

During the study, subjects will be evaluated for safety and toxicity, PK, efficacy, and predictive biomarkers.

### Part 1 Dose Escalation and Expansion Single Agent Therapy

Part 1 of the study will escalate Compound 1 as a single agent and will use the Bayesian optimal interval (BOIN) design for dose escalation. Compound 1 will be taken orally every other day in 28 day cycles. Subjects will be enrolled into 6 successive escalating dose level cohorts of 20 mg, 40 mg, 80 mg, 120 mg, 160 mg, and 200 mg of Compound 1 (see Table 1). DLT will be assessed during the first treatment cycle of 28 days. Dose escalation will occur only after all subjects have completed the DLT window of 28 days. Available safety data from subjects treated beyond the first cycle will also be taken into consideration for tolerability assessment.

Intra-subject dose escalation to the highest cleared dose level will be allowed for subjects enrolled on the lowest 3 dose levels in Part 1 and will start with their Cycle 3 if subjects did not experience >Grade 2 AEs in Cycles 1 and 2.

Dose escalation will continue until the MTD or the highest dose level, the maximum administered dose (MAD), is reached. Once the MTD or MAD is reached, the RP2D will be determined based on overall assessment of all safety data (including low grade but chronic toxicities, dose reductions, etc.), as well as all available PK, pharmacodynamic biomarker, and efficacy data collected during dose escalation.

TABLE 1
Part 1 Dose Levels of Compound 1

| Dose Level | Dose of Compound 1 (mg) |
|---|---|
| Level 1 | 20 |
| Level 2 | 40 |
| Level 3 | 80 |
| Level 4 | 120 |
| Level 5 | 160 |
| Level 6 | 200 |

### Part 1 Expansion Cohort:

Once Part 1 dose escalation is complete, the single agent RP2D of Compound 1 will be further tested in a Part 1 expansion cohort. The study plan is to enroll 20 subjects with platinum-resistant HGSOC (including primary peritoneal and fallopian tube cancer) or high-grade endometrial carcinoma (including uterine serous carcinoma and carcinosarcoma). Subjects must have locally advanced or metastatic non-resectable solid tumors with oncogene amplifications, whose disease has progressed despite all standard therapies, or for whom no further standard or clinically acceptable therapy exists.

Part 2 Dose Escalation of Compound 1 in Combination with Select Targeted Therapies

Part 2 of the study will escalate Compound 1 in combination with either erlotinib, pemigatinib, or abemaciclib according to matching oncogene amplification. The BOIN design will be applied for dose escalation.

The study plan is to enroll subjects to 3 combination escalation modules:
In Module 1, subjects will receive Compound 1 in combination with erlotinib. Subjects in Part 2 Module 1 must have evidence of EGFR amplification by Clinical Laboratory Improvement Amendments (CLIA)-certified NGS testing.
In Module 2, subjects will receive Compound 1 in combination with pemigatinib. Subjects in Part 2 Module 2 must have evidence of FGFR1, FGFR2, or FGFR3 amplification by CLIA-certified NGS testing.
In Module 3, subjects will receive Compound 1 in combination with abemaciclib. Subjects in Part 2 Module 3 must have evidence of CDK4 or CDK6 amplification by CLIA-certified NGS testing.

The dose level of Compound 1 from Part 1 will be determined from the ranges of about 10 mg to about 400 mg, including about 10 mg to 20 mg, about 20 mg to 40 mg, about 40 mg to about 80 mg, about 80 mg to about 120 mg, about 80 mg to about 200 mg, about 120 mg to about 160 mg, about 160 mg to about 200 mg, about 200 mg to about 400 mg, further including about 10 mg, about 20 mg, about 40 mg, about 80 mg, about 120 mg, about 160 mg, or about 200 mg for Part 2. The dose schedule of Compound 1 from Part 1 will be determined from the following options administered daily, every other day, on a cycle of day 1 and day 3 followed by a 4-day dosing holiday, every 3 days, weekly, or with a dosing holiday of 4 days, 4-7 days, or 14 days for Part 2.

Erlotinib, futibatinib, and pemigatinib will be administered at the doses per their respective USPIs; however, if significant toxicity occurs, these doses can be lowered for the study.

In Part 2 Module 3, both drugs will be escalated slowly and in a stepwise approach as specified in the study protocol. The starting dose level of Compound 1 will be at least 2 or more dose levels below the MTD or RP2D determined for single agent Compound 1 in Part 1 and will be determined. Exemplary dose levels of abemaciclib for the combination include 50, 100, or 150 mg taken orally twice daily in 28-day cycles. Subjects in Parts 2 and 3 with toxicities known to be directly associated with the combination treatment but are unrelated to Compound 1 (e.g., hyperphosphatemia with pemigatinib) that cause the discontinuation of the combination treatment may receive monotherapy with Compound 1 per Investigator discretion and remain on study. Subjects in combination treatment cohorts with toxicities related to Compound 1 who require discontinuation of Compound 1 will also discontinue the combination treatment.

The RP2Ds of Compound 1 in combination with either erlotinib, futibatinib, pemigatinib, or abemaciclib for Part 3 will be determined based on the overall assessment of all safety data, as well as all available PK, pharmacodynamic biomarker, and efficacy data obtained during Part 2 dose escalation. Available safety data beyond the first cycle will also be taken into consideration for tolerability assessment and RP2D determination. The determination of the RP2D will require that at least 6 subjects have been treated at the MTD or MAD.

Part 3 Dose Expansion of Compound 1 in Combination with Select Targeted Therapies

Part 3 of the study will evaluate Compound 1 in combination with either erlotinib, pemigatinib, or abemaciclib in 3 different combination modules with 1 expansion basket per Module. Part 3 will include approximately 23 to 40 subjects in each expansion Module as determined by the Simon's optimal two-stage design:
In Module 1, subjects will receive Compound 1 in combination with erlotinib. Subjects in Part 3 Module 1 must have evidence of ecDNA-enabled amplification of EGFR using ecDNA imaging technology.
In Module 2, subjects will receive Compound 1 in combination with pemigatinib or futibatinib. Subjects in Part 3 Module 2 must have evidence of ecDNA-enabled amplification of FGFR1, FGFR2, or FGFR3 using ecDNA imaging technology.
In Module 3 subjects will receive Compound 1 in combination with abemaciclib. Subjects in Part 3 Module 3 must have evidence of ecDNA-enabled amplification of CDK4 or CDK6 using ecDNA imaging technology.

Part 3 will begin enrolling after Part 2. Subjects in Part 3 must have evidence of ecDNA-enabled specific oncogene amplification (i.e., EGFR for Module 1; FGFR1, FGFR2, or FGFR3 for Module 2; and CDK4 or CDK6 for Module 3).

Subjects in Part 3 will be dosed with the RP2Ds of Compound 1 in combination with either erlotinib, pemigatinib (or futibatinib), or abemaciclib. Erlotinib and pemigatinib (or futibatinib) will be administered at doses per their respective USPIs or as determined in Part 2 if dose reductions were necessary for safety. Abemaciclib will be administered at its RP2D in combination with Compound 1 as determined from Part 2 Module 3. Subjects in Parts 2 and 3 with toxicities known to be directly associated with the combination treatment but unrelated to Compound 1 (e.g., hyperphosphatemia with pemigatinib) that cause the discontinuation of the combination treatment may receive monotherapy with Compound 1 per Investigator discretion and remain on study. Subjects in combination treatment cohorts with toxicities related to Compound 1 who require discontinuation of Compound 1 will also discontinue the combination treatment.

During Part 3, safety, laboratory, PK, pharmacodynamics, and efficacy imaging assessments will continue, and the RP2D will be confirmed or adjusted accordingly.

### Example 1b—an Open-Label, Multicenter, First-In-Human, Dose-Escalation and Dose-Expansion, Phase 1/2 Study of Compound 1 and Compound 1 in Combination with Select Targeted Therapies in Subjects with Locally Advanced or Metastatic Solid Tumors with Oncogene Amplifications

### Objectives

### Primary Objectives

To assess the safety, tolerability, and DLTs of Compound 1 as a single agent administered orally at escalating dose levels in adult subjects with locally advanced or metastatic solid tumors with oncogene amplifications.

To assess the safety, tolerability, and DLTs of Compound 1 in combination with each of the following agents: an EGFR inhibitor (e.g., erlotinib), an FGFR inhibitor (e.g., futibatinib), or a CDK4/6 inhibitor (e.g., abemaciclib), at escalating dose levels in adults with locally advanced or metastatic solid tumors with corresponding oncogene amplifications.

To determine the maximum tolerated dose (MTD) and the recommended Phase 2 dose (RP2D) of Compound 1 as a single agent.

To determine the MTD and the RP2D of Compound 1 in combination with each of the following agents: an EGFR inhibitor (e.g., erlotinib), an FGFR inhibitor (e.g., futibatinib), or a CDK4/6 inhibitor (e.g., abemaciclib).

### Secondary Objectives

To assess the PK of Compound 1 as a single agent and in combination with each of the following agents: an EGFR inhibitor (e.g., erlotinib), an FGFR inhibitor (e.g., futibatinib), or a CDK4/6 inhibitor (e.g., abemaciclib).

To assess the preliminary antitumor activity of Compound 1 as a single agent and in combination with each of the following agents: an EGFR inhibitor (e.g., erlotinib), an FGFR inhibitor (e.g., futibatinib), or a CDK4/6 inhibitor (e.g., abemaciclib).

### Exploratory Objectives

To assess predictive biomarkers for Compound 1 as a single agent and in combination with each of the following agents: an EGFR inhibitor (e.g., erlotinib), an FGFR inhibitor (e.g., futibatinib), or a CDK4/6 inhibitor (e.g., abemaciclib) (e.g., ecDNA enabled oncogene amplification).

To assess pharmacodynamic biomarkers for Compound 1 as a single agent and in combination with each of the following agents: erlotinib, pemigatinib, or abemaciclib.

To assess the PK of each of the following agents: an EGFR inhibitor (e.g., erlotinib), an FGFR inhibitor (e.g., futibatinib), or a CDK4/6 inhibitor (e.g., abemaciclib).

### Endpoints

### Primary Endpoints

TEAEs including determination of DLTs and SAEs. AEs will be assessed, and causality and severity will be assigned by using the National Cancer Institute Common Terminology Criteria for Adverse Events (NCI CTCAE), Version 5.0.

### Secondary Endpoints

PK parameters derived for Compound 1 as a single agent and in combination with each of an EGFR inhibitor (e.g., erlotinib), an FGFR inhibitor (e.g., futibatinib), or a CDK4/6 inhibitor (e.g., abemaciclib):
Plasma PK parameters (maximum plasma concentration [Cₘₐₓ], trough concentration [C_{trough}], time to maximum plasma concentration [Tₘₐₓ] and area under the concentration-time curve from 0 to last quantifiable concentration [AUC₀₋ₗₐₛₜ] on Day 1 and Day 15) will be estimated using standard non-compartmental PK analysis.
Other PK parameters (terminal half-life [t_{1/2}], clearance, and accumulation ratios based on Cₘₐₓ, C_{trough}, and AUC [RCₘₐₓ, RC_{trough}, and RC_{AUC}, respectively]) will be evaluated if data permit.

Clinical response including objective response rate (ORR), duration of response (DOR), disease control rate (DCR), progression-free survival (PFS), and overall survival (OS) to Compound 1 or Compound 1 in combination with each of select targeted therapies (i.e., an EGFR inhibitor (e.g., erlotinib), an FGFR inhibitor (e.g., futibatinib), or a CDK4/6 inhibitor (e.g., abemaciclib)), per RECIST v1.1.
ORR will be calculated as the sum of subjects with best overall response of complete response (CR) or partial response (PR).
DOR will be calculated as the time from earliest date of documented CR or PR until documented disease progression or death (by any cause, in the absence of progression). In progression-free subjects, DOR will be censored at the last evaluable tumor assessment following the earliest date of documented CR or PR.
DCR will be calculated as the sum of subjects with best overall response of CR, PR, or stable disease (SD).
PFS is defined as the time from start of study treatment, until documented disease progression or death (by any cause, in the absence of progression). In progression-free subjects, PFS will be censored at the last evaluable tumor assessment.
OS is defined as the time from start of study treatment, until death (by any cause) or date of censoring. Subjects alive or those lost to follow-up will be censored at the last date of contact (or last date known to be alive).

### Exploratory Endpoints

Potential predictive biomarkers for Compound 1 in tissue will be evaluated by NGS for cancer associated alterations and for ecDNA using ecDNA imaging technology; a subset of samples will be evaluated for oncogene amplification by fluorescent in situ hybridization (FISH). Plasma circulating tumor DNA (ctDNA) will be evaluated by NGS for oncogenic alterations and the presence of ecDNA.

### Overall Study Design

Compound 1 is an orally available, potent, and selective small molecule inhibitor of Chk1 intended as a tumor agnostic treatment for patients with locally advanced or metastatic solid tumors with oncogene amplification on ecDNA. This is a First in Human (FIH), open-label, non-randomized, 3-part, Phase 1/2 study to determine the safety profile and identify the MTD and RP2D of Compound 1 administered as a single agent or in combination with a select targeted therapy, i.e., an EGFR inhibitor (e.g., erlotinib), an FGFR inhibitor (e.g., futibatinib), or a CDK4/6 inhibitor (e.g., abemaciclib). Compound 1 will be administered orally to subjects with locally advanced or metastatic non-resectable solid tumors harboring oncogene amplifications, whose disease has progressed despite all standard therapies or for whom no further standard or clinically acceptable therapy exists.

Part 1 is a single agent Compound 1 dose escalation and dose expansion; Part 2 is a dose escalation of Compound 1 in combination with either an EGFR inhibitor (e.g., erlotinib), an FGFR inhibitor (e.g., futibatinib), or a CDK4/6 inhibitor (e.g., abemaciclib); and Part 3 will be a dose expansion of Compound 1 in combination with either an EGFR inhibitor (e.g., erlotinib), an FGFR inhibitor (e.g., futibatinib), or a CDK4/6 inhibitor (e.g., abemaciclib).

Subjects recruited for any part of the study will provide informed consent and will undergo Screening before study participation. After a Screening period, qualified subjects will be enrolled to receive their assigned dose regimen of Compound 1 as a single agent or in combination with either an EGFR inhibitor (e.g., erlotinib), an FGFR inhibitor (e.g., futibatinib), or a CDK4/6 inhibitor (e.g., abemaciclib). In all parts of the study, subjects will be treated until progression of disease, development of unacceptable toxicity, or withdrawal from the study or withdrawal of consent, whichever occurs first.

During the study, subjects will be evaluated for safety and toxicity, PK, efficacy, and predictive biomarkers.

### Part 1 Dose Escalation and Expansion Single Agent Therapy

Part 1 of the study will escalate Compound 1 as a single agent and will use the Bayesian optimal interval (BOIN) design for dose escalation. Compound 1 will be taken orally in 28 day cycles. DLT will be assessed during the first treatment cycle of 28 days. Dose escalation will occur only after all subjects have completed the DLT window of 28 days. Available safety data from subjects treated beyond the first cycle will also be taken into consideration for tolerability assessment.

Dose escalation will continue until the MTD or the highest dose level, the maximum administered dose (MAD), is reached. Once the MTD or MAD is reached, the RP2D will be determined based on overall assessment of all safety data (including low grade but chronic toxicities, dose reductions, etc.), as well as all available PK, pharmacodynamic biomarker, and efficacy data collected during dose escalation.

### Part 1 Expansion Cohort:

Once Part 1 dose escalation is complete, the single agent RP2D of Compound 1 will be further tested in a Part 1 expansion cohort. The study plan is to enroll 20 subjects with platinum-resistant HGSOC (including primary peritoneal and fallopian tube cancer) or high-grade endometrial carcinoma (including uterine serous carcinoma and carcinosarcoma). Subjects must have locally advanced or metastatic non-resectable solid tumors with oncogene amplifications, whose disease has progressed despite all standard therapies, or for whom no further standard or clinically acceptable therapy exists.

Part 2 Dose Escalation of Compound 1 in Combination with Select Targeted Therapies

Part 2 of the study will escalate Compound 1 in combination with either erlotinib, futibatinib, or abemaciclib according to matching oncogene amplification. The BOIN design will be applied for dose escalation.

The study plan is to enroll subjects to 3 combination escalation modules:
In Module 1, subjects will receive Compound 1 in combination with erlotinib. Subjects in Part 2 Module 1 must have evidence of EGFR amplification by Clinical Laboratory Improvement Amendments (CLIA)-certified NGS testing.
In Module 2, subjects will receive Compound 1 in combination with futibatinib. Subjects in Part 2 Module 2 must have evidence of FGFR1, FGFR2, FGFR3 or FGFR4 amplification by CLIA-certified NGS testing.
In Module 3, subjects will receive Compound 1 in combination with abemaciclib. Subjects in Part 2 Module 3 must have evidence of CDK4 or CDK6 amplification by CLIA-certified NGS testing.

The dose level of Compound 1 from Part 1 will be determined from the ranges of about 10 mg to about 400 mg, including about 10 mg to 20 mg, about 20 mg to 40 mg, about 40 mg to about 80 mg, about 80 mg to about 120 mg, about 80 mg to about 200 mg, about 120 mg to about 160 mg, about 160 mg to about 200 mg, about 240 mg, about 200 mg to about 400 mg, further including about 10 mg, about 20 mg, about 40 mg, about 80 mg, about 120 mg, about 160 mg, about 200 mg or about 240 mg for Part 2. The dose schedule of Compound 1 from Part 1 will be determined from the following options administered (1) every other day, (2) on a cycle of day 1 and day 2 followed by a 5-day dosing holiday, (3) every 3 days, (4) weekly, (5) on a cycle of days 1, 2, and 3, followed by an 11-day dosing holiday, or (6) with a dosing holiday of 4 days, 5 days, 4-7 days, 11 days, or 14 days for Part 2.

Erlotinib and futibatinib, will be administered at the doses per their respective USPIs; however, if significant toxicity occurs, these doses can be lowered for the study.

In Part 2 Module 3, both drugs will be escalated slowly and in a stepwise approach as specified in the study protocol. The starting dose level of Compound 1 will be at least 2 or more dose levels below the MTD or RP2D determined for single agent Compound 1 in Part 1 and will be determined. Exemplary dose levels of abemaciclib for the combination include 50, 100, or 150 mg taken orally twice daily in 28-day cycles.

The RP2Ds of Compound 1 in combination with either erlotinib, futibatinib, or abemaciclib for Part 3 will be determined based on the overall assessment of all safety data, as well as all available PK, pharmacodynamic biomarker, and efficacy data obtained during Part 2 dose escalation. Available safety data beyond the first cycle will also be taken into consideration for tolerability assessment and RP2D determination.

Part 3 Dose Expansion of Compound 1 in Combination with Select Targeted Therapies

Part 3 of the study will evaluate Compound 1 in combination with either erlotinib, futibatinib, or abemaciclib in 3 different combination modules with 1 expansion basket per Module. Part 3 will include approximately 23 to 40 subjects in each expansion Module as determined by the Simon's optimal two-stage design:
In Module 1, subjects will receive Compound 1 in combination with erlotinib. Subjects in Part 3 Module 1 must have evidence of ecDNA-enabled amplification of EGFR.
In Module 2, subjects will receive Compound 1 in combination with futibatinib. Subjects in Part 3 Module 2 must have evidence of ecDNA-enabled amplification of FGFR1, FGFR2, FGFR3 or FGRF4 using ecDNA imaging technology.
In Module 3 subjects will receive Compound 1 in combination with abemaciclib. Subjects in Part 3 Module 3 must have evidence of ecDNA-enabled amplification of CDK4 or CDK6 using ecDNA imaging technology.

Part 3 will begin enrolling after Part 2. Subjects in Part 3 must have evidence of ecDNA-enabled specific oncogene amplification (i.e., EGFR for Module 1; FGFR1, FGFR2, FGFR3, or FGFR4 for Module 2; and CDK4 or CDK6 for Module 3).

Subjects in Part 3 will be dosed with the RP2Ds of Compound 1 in combination with either erlotinib, futibatinib, or abemaciclib. Erlotinib and futibatinib will be administered at doses per their respective USPIs or as determined in Part 2 if dose reductions were necessary for safety. Abemaciclib will be administered at its RP2D in combination with Compound 1 as determined from Part 2 Module 3. During Part 3, safety, laboratory, PK, pharmacodynamics, and efficacy imaging assessments will continue, and the RP2D will be confirmed or adjusted accordingly.

### Example 2—Combination of Compound 1 and an EGFR Inhibitor in a NSCLC Squamous Cell Carcinoma Model

In a preclinical patient-derived xenografts (PDX) NSCLC squamous cell carcinoma model with ecDNA-enabled amplification of wildtype EGFR, Compound 1 has shown synergistic combinatorial antitumor activity with erlotinib. The LU1206 PDX model harbors high basal ecDNA-enabled copy number amplification of EGFR and shows marginal antitumor response to erlotinib (dosed orally at 50 mg/kg once per day) and Compound 1 (dosed orally at 50 mg/kg every other day) monotherapy. The combination therapy of Compound 1 plus erlotinib showed synergistic antitumor activity when compared with either monotherapy alone and resulted in a significant delay in tumor growth. These findings demonstrate the potential ability of Compound 1 plus erlotinib combination therapy to have significant antitumor activity in cancers with ecDNA-enabled amplifications of wildtype EGFR.

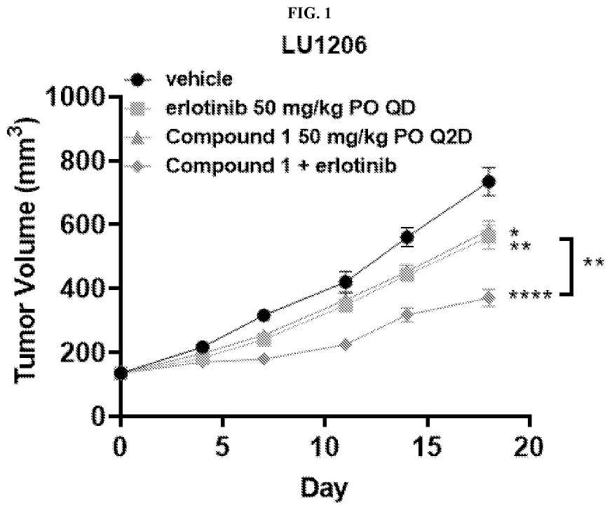

The anti-tumor activity and PD of single agent Compound 1, single agent EGFR inhibitor erlotinib, and combination of Compound 1 plus erlotinib were evaluated in a ecDNA+ EGFR amplified NSCLC squamous cell carcinoma LU1206 PDX model. For single agent and combination arms, Compound 1 was dosed at 50 mg/kg PO Q2D and erlotinib was dosed at 50 mg/kg PO QD when average tumor volumes reached 140 mm³ in female BALB/c nude mice, n=10 mice/group (FIG. 1). Treatment with single agent Compound 1 or single agent erlotinib and the combination therapy showed modest delay in tumor growth that was significant when compared to vehicle on Study Day 18, with mean % ΔTGI of 25.3%, 28.9%, and 60.7%, respectively. Additionally, the anti-tumor activity of the combination therapy was significantly better than that of each single agent alone. The combination of Compound 1 plus erlotinib was determined to be synergistic when compared to respective single agent arms using the Fractional Product Method (Webb, 1963); observed fu=0.5047<calculated fu=0.6067 (FIG. 1). These results demonstrate that Compound 1 in combination with erlotinib is superior to either single agent Compound 1 or single agent erlotinib and resulted in synergistic anti-tumor activity in a tumor model harboring oncogene amplification of EGFR on ecDNA. Anti-tumor activity of single agent Compound 1 (triangle), single agent erlotinib (square), and Compound 1 in combination with erlotinib (diamond) were evaluated in the ecDNA+ EGFR amplified LU1206 PDX model. Tumor volume (mm³) data are shown as mean±SEM for each treatment group. Significant anti-tumor activity was observed with the combination therapy on Day 18 when compared with vehicle. Significance between groups was determined by ordinary one-way ANOVA with Tukey's multiple comparisons using GraphPad Prism software, *p-value<0.05, **p-value<0.001, ****p-value<0.0001. PO: orally, QD: once per day, Q2D: once every other day.

### Example 3—Combination of Compound 1 and an FGFR Inhibitor in a Gastric Adenocarcinoma Model

In a preclinical cell line-derived xenograft (CDX) gastric adenocarcinoma model with ecDNA-enabled amplification of FGFR2, Compound 1 has shown synergistic combinatorial antitumor activity with the FGFR1-3 inhibitor infigratinib. The SNU-16 CDX model develops acquired resistance to infigratinib monotherapy via increases in FGFR2 copy number on ecDNA to out-titrate PK levels of infigratinib at its maximally tolerated dose.

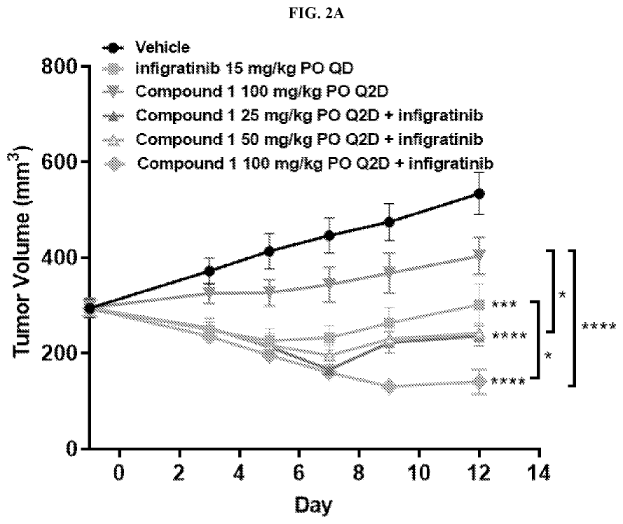

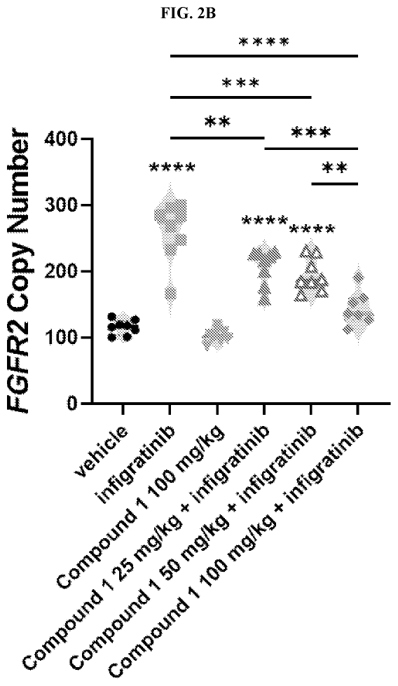

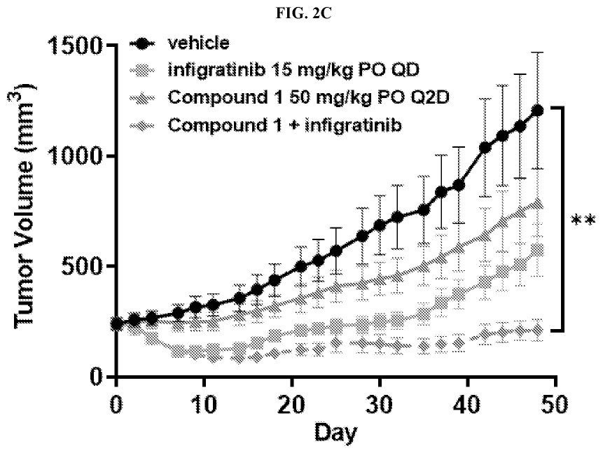

The activity of Compound 1, as a single agent or in combination with infigratinib was assessed by measures of anti-tumor and PD activity in multiple studies using the SNU-16 CDX tumor model. In the first study female SCID beige mice were dosed with Compound 1 at 100 mg/kg PO Q2D or infigratinib at 15 mg/kg PO QD as a single agent or with Compound 1 at 25, 50, or 100 mg/kg PO Q2D in combination with infigratinib, dosed at 15 mg/kg PO QD, when average tumor volumes reached 350 mm³ (n=8 animals per group). As shown in FIG. 2A, single agent Compound 1 at 100 mg/kg resulted in slight tumor growth delay with mean % ΔTGI of 54.8% that was not significant when compared to tumors from the vehicle arm on Day 12. Single agent infigratinib significantly delayed tumor growth when compared to tumors from the vehicle arm on Day 12 with a mean % ΔTGI of 97.6%. However, though SNU-16 CDX tumors initially demonstrated an anti-tumor response to single agent infigratinib, after approximately 1 week of continuous treatment, tumors resumed growth along with a concomitant increase in FGFR2 gene copy number detected by 12 days following initiation of infigratinib treatment (FIG. 2B). This observation supports a potential role for ecDNA+ FGFR2 copy number increase in acquired resistance to single agent infigratinib. The combination of Compound 1, at all dose levels, plus infigratinib resulted in significant TGI and tumor regressions when compared to tumors from vehicle treated animals, with mean % ΔTGI of 123%, 121%, and 164%, for 25, 50, and 100 mg/kg Compound 1 plus infigratinib, respectively. Additionally, the combination therapy of Compound 1 (100 mg/kg) plus infigratinib showed significantly better anti-tumor activity than infigratinib single agent, with mean % DTGI of 164.1% and 97.6%, respectively. The combination therapy of Compound 1 at 100 mg/kg plus infigratinib at 15 mg/kg was determined to be synergistic when compared to respective single agent arms using the Fractional Product Method (Webb, 1963); observed fraction unaffected (fu)=0.2628<calculated fu=0.4266. Additionally, a Compound 1 dose-dependent inhibition of infigratinib treatment-induced FGFR2 copy number increase was observed in tumors evaluated from the combination therapy arms (FIG. 2B). These findings demonstrate that combination of Compound 1 plus infigratinib can attenuate increases in FGFR2 copy number induced by FGFR targeted therapy and result in synergistic anti-tumor activity that is superior to single agent Compound 1 or single agent infigratinib in tumors harboring oncogene amplification of FGFR2 on ecDNA.

In subsequent a study, the durability of the anti-tumor response of Compound 1 plus infigratinib combination therapy was also assessed in the SNU-16 CDX tumor model. Female BALB/c nude mice were dosed with single agent Compound 1 at 50 mg/kg PO Q2D or in combination with infigratinib, dosed at 15 mg/kg PO QD, when average tumor volume reached 300 mm³ (n=10 animals per group). As shown in FIG. 2C, single agent infigratinib or single agent Compound 1 at 50 mg/kg resulted in modest tumor growth delays with mean % ΔTGI of 65.4% and 43.1%, respectively, that was not significant compared to tumors from vehicle treated animals. In contrast, the combination of Compound 1 at 50 mg/kg plus infigratinib resulted in significant TGI when compared to tumors from vehicle treated animals with a mean % ΔTGI of 102% that was durable and observed on Day 48 of treatment. The combination of Compound 1 at 50 mg/kg plus infigratinib was determined to be synergistic when compared to respective single agent arms using the Fractional Product Method (Webb, 1963); observed fu=0.1754<calculated fu=0.3108. These results demonstrate that combination therapy of Compound 1 plus infigratinib is superior to either single agent alone and can result in synergistic and durable anti-tumor activity in tumors harboring oncogene amplification of FGFR2 on ecDNA. The anti-tumor activity of single agent Compound 1 (triangle), single agent infigratinib (square), and Compound 1 in combination with infigratinib at various dose and schedules were evaluated in the ecDNA+ FGFR2 amplified SNU-16 CDX model. Tumor volume (mm³) data are shown as mean z SEM for each treatment group. A) The combination therapy arms showed significant anti-tumor response when compared to vehicle on Day 12. Additionally, the combination therapy arm of Compound 1 at 100 mg/kg showed significant anti-tumor response when compared to either single agent arm alone. FGFR2 copy number was evaluated by qPCR on DNA isolated from tumors treated for 12 days with single agent Compound 1, single agent infigratinib, or the combination of both agents. The combination therapy showed a significant and dose dependent reduction in tumor FGFR2 copy number when compared to infigratinib single agent. *p-value<0.05, **p-value<0.01, ***p<0.001, ****p-value<0.0001. B) Significant and durable anti-tumor activity was also observed with combination of infigratinib plus Compound 1 at 50 mg/kg compared to vehicle. **p-value<0.01. Significance between groups was determined by ordinary one-way ANOVA with Tukey's multiple comparisons using GraphPad Prism software. PO: orally, mpk: mg/kg, QD: once per day, Q2D: once every other day.

### Example 4—Combination of Compound 1 and CDK4/6 Inhibitors in a Osteosarcoma Model

In a preclinical cell line-derived xenograft (CDX) osteosarcoma model with ecDNA-enabled amplification of wildtype CDK4, Compound 1 has shown synergistic combinatorial antitumor activity with the CDK4/6 inhibitors abemaciclib and palbociclib. The SJSA-1 CDX model harboring ecDNA-enabled wildtype CDK4 amplification shows marginal antitumor response to abemaciclib (dosed orally at 10.5 mg/kg once every day) and palbociclib (dosed orally at 20 mg/kg once every day) monotherapy. Palbociclib monotherapy at 50 mg/kg dosed orally every day resulted in transient stasis of tumor growth followed by acquired therapeutic resistance after 15 days. The combination therapy of Compound 1 (dosed orally at 50 mg/kg every other day) with abemaciclib (dosed orally at 10.5 mg/kg once per day) demonstrated synergistic antitumor effect when compared with either monotherapy alone. Additionally, combination therapy of Compound 1 (dosed orally at 50 mg/kg every other day) and palbociclib (dosed orally at 50 mg/kg once per day) demonstrated a synergistic antitumor effect when compared to either monotherapy alone at respective doses and resulted in a significant delay in tumor growth. These findings demonstrate the potential ability of Compound 1 plus targeted CDK4/6 combination therapy to have significant antitumor activity in cancers with ecDNA-enabled amplification of wildtype CDK4/6.

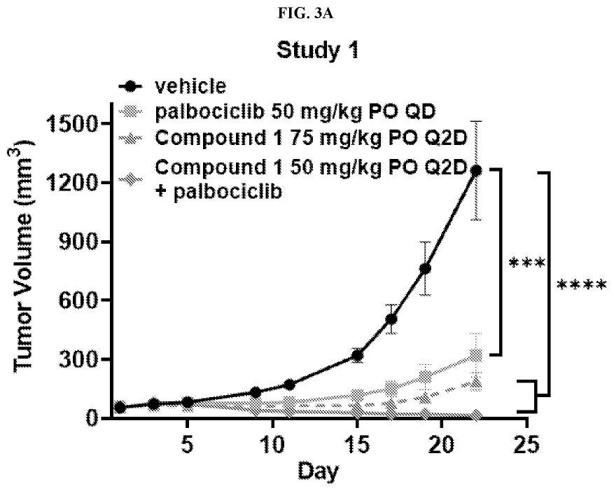

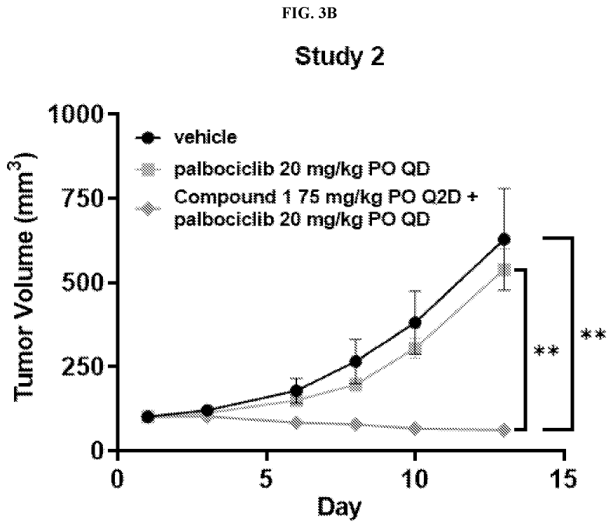

The anti-tumor activity and PD of Compound 1 in combination with the CDK4/6 inhibitor palbociclib was determined in the ecDNA+ CDK4 amplified osteosarcoma SJSA-1 CDX tumor model. Female Hsd:Athymic Nude-Foxnlnu mice were dosed with single agent Compound 1 at 50 or 75 mg/kg PO Q2D, and single agent palbociclib was dosed at 20 or 50 mg/kg PO QD (n=8 animals per group). For combination therapy assessment, palbociclib was dosed at 50 mg/kg PO QD and Compound 1 was dosed at 50 mg/kg PO Q2D, or palbociclib was dosed at 20 mg/kg PO QD and Compound 1 was dosed at 75 mg/kg PO Q2D (n=8 animals per group). Treatment was initiated when average tumor volumes reached 56.5 or 96.2 mm³ (FIG. 3A and FIG. 3B). Treatment with single agent Compound 1 at 50 mg/kg resulted in slight TGI that was not significant when compared to tumors from vehicle treated animals. Similarly, single agent palbociclib at 20 mg/kg resulted in slight anti-tumor activity, with a mean % ΔTGI of 16.9%, that was not significant when compared to tumors from vehicle treated animals (FIG. 3B). Single agent palbociclib at 50 mg/kg resulted in significant anti-tumor activity when compared to vehicle, with mean % ΔTGI of 78.3% (FIG. 3A). The combination of Compound 1 plus palbociclib at each of the various dose levels evaluated resulted in significant anti-tumor activity, including tumor regressions, when compared to vehicle, with mean % ΔTGI of 103% for Compound 1 (50 mg/kg) plus palbociclib (50 mg/kg), and mean % ΔTGI of 107% for Compound 1 (75 mg/kg) plus palbociclib (20 mg/kg). These findings demonstrated that Compound 1 combination therapy with palbociclib is superior to either palbociclib or Compound 1 single agent alone and can lead to durable anti-tumor responses, including tumor regressions, in tumors harboring oncogene amplification of CDK4 on ecDNA. The anti-tumor activity of single agent Compound 1 (triangle), single agent palbociclib (square), and Compound 1 in combination with palbociclib at indicated doses and schedules (diamond) were evaluated in the ecDNA+ CDK4 amplified SJSA-1 CDX tumor model. Tumor volume (mm³) data are shown as mean±SEM for each treatment group. For Study 1 (FIG. 3A), dosing was initiated when tumor volumes reached 56.5+/−SD 8.9 mm³. On Day 22, significant anti-tumor activity was observed with combination therapy, including regressions. For Study 2 (FIG. 3B), dosing was initiated when tumor volumes reached or 96.2+/−SD 13.7 mm³. On Day 13, significant anti-tumor activity was observed with combination therapy when compared with vehicle and single agent palbociclib. For both studies, significance was determined by ordinary one-way ANOVA with Tukey's multiple comparisons using GraphPad Prism software. **p-value<0.005, ***p-value<0.001, ****p-value<0.0001. PO: orally, QD: once every day, Q2D: once every other day.

### Example 5—Preliminary Pharmacokinetic (PK) Data Analysis of Subjects Dosed with Compound 1

Pharmacokinetic (PK) data analysis of the first eleven subjects treated with Compound 1 on clinical study BBI-355-101 revealed a longer half-life than predicted based on preclinical PK data (i.e., approximately 47 hours observed in humans) and an accumulation ratio of ~2.9, leading to continuous daily drug exposure of Compound 1.

Preliminary PK data (i.e., half-life) of the first eleven subjects treated with Compound 1 orally every other day (Q2D) is shown in Table 2. The mean half-lives ranged from 42 to 62 hours. Comparison between Day 1 and Day 15 concentration-time profiles showed an increase accumulation in plasma Compound 1 after PO Q2D administration. The observed human prolonged half-life and accumulation ratio (~2.9) differed from what was predicted from preclinical studies.

TABLE 2
Human half-life of Compound 1 in Subjects Dosed at 20 mg, 40 mg, and
80 mg Orally, Q2D at Cycle 1 Day 1 and Day 15

| Cycle/Day | Cycle 1 Day 1 | | | Cycle 1 Day 15 | |
|---|---|---|---|---|---|
| Dose Cohort | 20 mg Q2D | 40 mg Q2D | 80 mg Q2D | 20 mg Q2D | 40 mg Q2D |
| PK Parameters | Mean ± SD | Mean ± SD | Mean ± SD | Mean ± SD | Mean ± SD |
| t_{1/2} (h) | 62 ± 46 | 42 ± 46 | 50 ± 32 | 45 ± 20 | 47 ± 13 |

A PK model was developed incorporating human Compound 1 PK data, with the goal to identify a potentially safer less frequent dosing schedule. Based on this model, the dosing schedule was adjusted to evaluate Compound 1 dosed 2ON/5OFF (daily for 2 days followed by 5 days off) repeated weekly. The intermittent 2ON/5OFF dosing schedule was selected to provide longer breaks in Compound 1 pharmacologic exposure in normal tissue to minimize toxicities, while maintaining sufficient drug exposure to provide potential anti-cancer activity.

On the Compound 1 dosing schedule of 2ON/5OFF, subjects will be enrolled into 5 successive escalating dose level cohorts of 80 mg, 120 mg, 160 mg, 200 mg, and 240 mg of Compound 1. If the 2ON/5OFF schedule is not tolerated, the administration of Compound 1 will be once weekly. Table 3 shows the doses and dosing schedules.

TABLE 3
Doses and Dosing Schedules of Compound 1

| | |
|---|---|
| Dose of Compound 1 (mg) | Compound 1 Dosing Schedule |

Dosing Schedule: Compound 1 every other day (Q2D)

| | |
|---|---|
| 20 | Every other day (Q2D) |
| 40 | Every other day (Q2D) |
| 80 | Every other day (Q2D) |

Dosing Schedule: Compound 1 daily for 2 days
followed by 5 days off (2ON/5OFF) repeated weekly

| | |
|---|---|
| 80 | 2 days ON/5 days OFF (2ON/5OFF) |
| 120 | 2 days ON/5 days OFF (2ON/5OFF) |
| 160 | 2 days ON/5 days OFF (2ON/5OFF) |
| 200 | 2 days ON/5 days OFF (2ON/5OFF) |
| 240 | 2 days ON/5 days OFF (2ON/5OFF) |

Dosing Schedule: Compound 1 once weekly (QW)

| | |
|---|---|
| 80 | Once weekly (QW) |
| 120 | Once weekly (QW) |
| 160 | Once weekly (QW) |
| 200 | Once weekly (QW) |
| 240 | Once weekly (QW) |

### Example 6. Monotherapy Anti-Tumor Activity of Compound 1

In vivo anti-tumor activity and tolerability of Compound 1 alone at different oral doses and schedules was assessed in the cell line derived xenograft (CDX) Kelly representing MYCN amplified neuroblastoma. For this study, mice were implanted with the Kelly tumor cells; once the tumors reached a volume of about 149 mm³, the mice were started on vehicle or single agent Compound 1 with the following doses and schedules: vehicle PO dosed QD for 24 days; Compound 1 at 50 mg/kg PO dosed Q2D) for 5 continuous weeks or 2 weeks on, 1 week off, 2 weeks on; Compound 1 at 5 or 10 mg/kg PO dosed QD for 28 days; Compound 1 at 100 or 200 mg/kg PO dosed Q7D) for 4 weeks. For comparison, the CHK1 inhibitor prexasertib was dosed at 15 mg/kg SC Q14D, based on its clinically relevant dose, route of administration and schedule (the human recommended Phase 2 dose for prexasertib is 105 mg/m2 administered by IV Q14D).

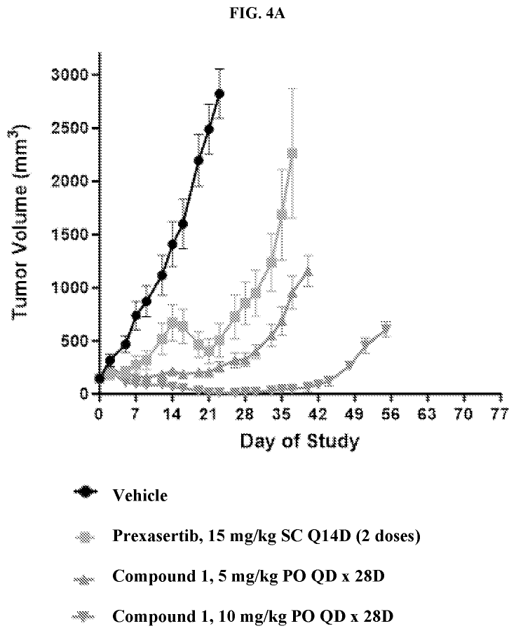

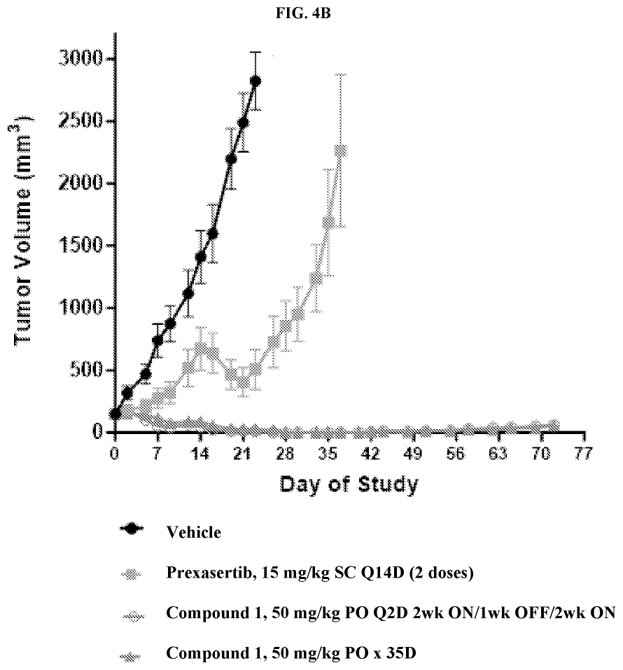

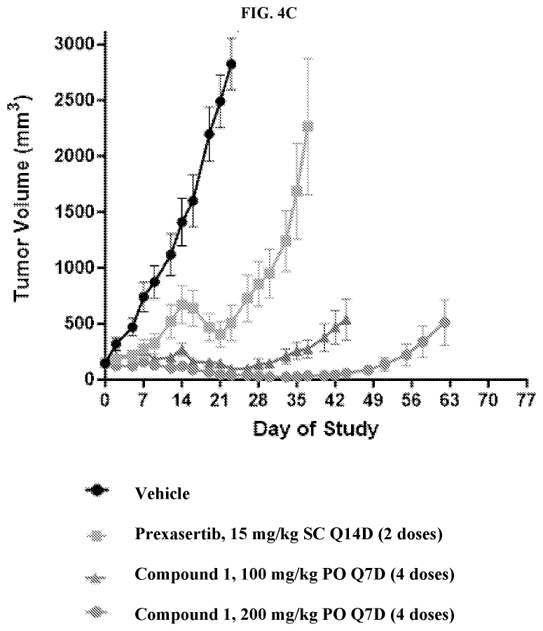

Results are shown in FIG. 4A, FIG. 41B, and FIG. 4C. All single agent Compound 1 doses and schedules resulted in significant anti-tumor activity (p<0.0001 by one-way ANOVA with Tukey's multiple comparisons test)), including tumor regressions, when compared to vehicle on day 23, the last day all vehicle tumors were on study. In addition, all Compound 1 treatment arm resulted in mean tumor volumes significantly smaller (p<0.05-p<0.001) than the mean tumor volume of the prexasertib arm when compared by unpaired two-tailed tests. A comparison of the mouse PK parameters in shown in Table 4.

TABLE 4
Mouse PK Paramters

| Compound and dosing | Cₘₐₓ (ng/ml) | AUC_{inf} (hr*ng/ml) |
|---|---|---|
| Prexasertib SC 15 mg/kg | 1030 | 2775 |
| Compound 1 PO 50 mg/kg | 144 | 1261 |

The examples and embodiments described herein are for illustrative purposes only and in some embodiments, various modifications or changes are to be included within the purview of disclosure and scope of the appended claims.

What is claimed is:

1. A method of treating cancer in a subject in need thereof comprising administering to the subject a pharmaceutical composition comprising 5-((5-(4-(((1R,3S)-3-aminocyclopentyl)oxy)-2-methoxy-6-methylpyridin-3-yl)-1H-pyrazol-3-yl)amino)pyrazine-2-carbonitrile: 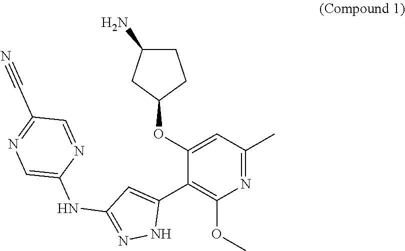 [SMILES: COc1nc(C)cc(O[C@@H]2CC[C@H](N)C2)c1-c1cc(Nc2cnc(C#N)cn2)n[nH]1]
or a pharmaceutically acceptable salt thereof, at a dose of about 10 mg to about 400 mg, whereby the subject experiences a therapeutic response.

2. The method of claim 1, wherein the dose of Compound 1 is between about 10 mg to about 20 mg, about 20 mg to about 40 mg, about 40 mg to about 80 mg, about 80 mg to about 120 mg, about 120 mg to about 160 mg, about 160 mg to about 200 mg, about 200 mg to about 240 mg, about 200 mg to about 400 mg.

3. The method of claim 1, wherein the dose of Compound 1 is 10 mg, about 20 mg, about 40 mg, about 80 mg, about 120 mg, about 160 mg, about 200 mg, or about 240 mg.

4. The method of any one of claims 1-3, wherein the cancer comprises a solid tumor.

5. The method of claim 4, wherein the cancer comprises a locally advanced or metastatic non-resectable solid tumor.

6. The method of any one of claims 1-5, wherein the cancer comprises a tumor or tumor cells harboring an oncogene amplification.

7. The method of claim 6, wherein the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FGFR4, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

8. The method of claim 6 or claim 7, wherein the oncogene amplification resides on ecDNA.

9. The method of claim 6 or claim 7, wherein the oncogene amplification resides on one or more chromosomal loci.

10. The method of claim 6 or claim 7, wherein the oncogene amplification is an ecDNA-derived amplification.

11. The method of any one of claims 6-10, wherein the oncogene amplification has a copy number of at least 6, at least 8, at least 10, at least 15, at least 20, or more than 20 copies of the oncogene or portion thereof.

12. The method of any one of claims 1-11, wherein the subject has undergone one or more prior therapies.

13. The method of claim 12, wherein the subject was non-responsive to the one or more prior therapies.

14. The method of claim 12, wherein the subject developed resistance to the one or more prior therapies.

15. The method of any one of claims 1-14, wherein the composition is administered orally.

16. The method of any one of claims 1-15, wherein the composition is administered every other day.

17. The method of any one of claims 1-15, wherein the composition is administered on a cycle of day 1 and day 3 followed by a 4 day dosing holiday.

18. The method of any one of claims 1-15, wherein the composition is administered every 3 days or weekly.

19. The method of any one of claims 1-15, wherein the composition is administered with a dosing holiday of 4 days, 4-7 days, 5 day, 7 days, 11 days, or 14 days.

20. The method of any one of claims 1-15, wherein the composition is administered on a cycle of day 1 and day 2 followed by a 5 day dosing holiday.

21. The method of any one of claims 1-15, wherein the composition is administered once weekly.

22. The method of any one of claims 1-15, wherein the composition is administered on a cycle of days 1, 2 and 3, followed by an 11 day dosing holiday.

23. The method of any one of claims 1-15, wherein the dose of Compound 1 about 80 mg, about 120 mg, about 160 mg, about 200 mg, or about 240 mg, and the composition is administered on a cycle of day 1 and day 2 followed by a 5 day dosing holiday.

24. The method of any one of claims 1-15, wherein the dose of Compound 1 about 80 mg, about 120 mg, about 160 mg, about 200 mg, or about 240 mg, and the composition is administered once weekly.

25. The method of any one of claims 1-24, wherein the cancer is bladder cancer, breast cancer, endometrial cancer, esophageal cancer, gastric cancer, head and neck squamous cell carcinoma, liposarcoma, non-small cell lung adenocarcinoma, non-small cell lung cancer, non-small cell squamous lung cancer, or ovarian cancer.

26. The method of any one of claims 1-24, wherein the cancer is ovarian cancer.

27. The method of claim 26, wherein the ovarian cancer is a platinum resistant high-grade serous ovarian cancer, a primary peritoneal cancer, or a fallopian tube cancer.

28. The method of any one of claims 1-24, wherein the cancer is a uterine cancer.

29. The method of claim 28, wherein the uterine cancer is a high-grade endometrial carcinoma, a uterine serous carcinoma, or a uterine carcinosarcoma.

30. The method of any one of claims 1-24, wherein the cancer is a neuroblastoma.

31. The method of any one of claim 1-30, wherein the treatment further comprises administering an additional therapeutic agent.

32. The method of claim 31, wherein the oncogene amplification comprises a CDK4, CDK6, EGFR, FGFR1, FGFR2, FGFR3, or FGFR4 amplification.

33. The method of claim 32, wherein the oncogene amplification comprises a wildtype FGFR1, FGFR2, FGFR3, or FGFR4 amplification.

34. The method of claim 32, wherein the oncogene amplification comprises a wildtype EGFR amplification.

35. The method of claim 30 or 31, wherein the oncogene amplification comprises an amplification of EGFR and wherein the treatment further comprises administering an EGFR inhibitor.

36. The method of claim 35, wherein the application of EGFR is an amplification of a wildtype EGFR.

37. The method of claim 35 or 36, wherein the EGFR inhibitor is selected from the group consisting of abivertinib, ABX-900, afatinib, agerafenib (RXDX-105), alflutinib mesylate, amivantamab, APL-1898, ASK-120067, aumolertinib (almonertinib), BBT-176, BDTX-1535, BDTX-189, BEBT-109, befortinib mesylate, beitatini, BLU-701, BLU-945, BPI-361175, BPI-7711, BPI-D0316, C-005, CDP1, cetuximab, CH-7233163, CK-101, CMAB-017, dacomitinib, depatuxizumab, depatuxizumab mafodotin (ABT-414), DFP-17729, dositinib, DS-2087, DZD-9008, E01001, E-TOC, epertinib, epitinib (HMPL-813), erlotinib, ES-072, FCN-411, FHND-9041, furmonertinib, FWD-1509, GB-263, GC-1118A, gefitinib, GMA-204, GR-1401, Hemay-022, HLX-07, HS-627, 1-010, icotinib, imgatuzumab, IN-A008, JMT-101, JRF-103, JS-111, JS-113, JZB-28, KN-023, KN-026, KP-673, lapatinib, larotinib, lazertinib, LL-191, LYN 205, M1231, maihuatinib, marizomib, mobocertinib, MP-0274, MRG003, naputinib tosilate, nazartinib, necitumumab, neptinib, nimotuzumab, NRC-2694-A, NT-004, OBX1-012, olafertinib, olmutinib, ORIC-114, oritinib, osimertinib, panitumumab, pirotinib, poziotinib, PRB-001, pyrotinib, QL-1203, SCT-200, serclutamab, SHR-A1307, SIM-200, SPH-1188, SSGJ-612, SYN-004, TAD-011, tarloxotinib, TAS-6417, TGRX-360, theliatinib (HMPL-309), TPC-064, TQB-3804, TY-9591, WJ-13404, WSD-0922, XZP-5809, yinlitinib maleate, YK-029A, YZJ-0318, zorifertinib, and ZSP-0391.

38. The method of claim 35 or 36, wherein the EGFR inhibitor is erlotinib.

39. The method of claim 38, wherein the erlotinib is administered to the subject at a dose of 150 mg PO daily or 100 mg PO daily or 50 mg PO daily.

40. The method of claim 39, wherein the dose of Compound 1 is between about 20 to about 40 mg, about 40 to about 80 mg, about 80 to about 120 mg, about 120 mg to about 160 mg, or about 160 mg to about 200 mg.

41. The method of any one of claims 35-40, wherein the cancer is colorectal cancer, esophageal cancer, gastric cancer, gastroesophageal junction (GEJ) cancer, head and neck squamous cell carcinoma, non-small cell lung cancer, or subtype squamous cell carcinoma.

42. The method of claim 30 or 31, wherein the oncogene amplification comprises an amplification of FGFR1, FGFR2, FGFR3, FGFR4, or a combination thereof and wherein the treatment further comprises administering an FGFR inhibitor.

43. The method of claim 42, wherein the application of FGFR1, FGFR2, FGFR3 or FGFR4 is an amplification of a wildtype FGFR1, FGFR2, FGFR3 or FGFR4.

44. The method of claim 42 or 43, wherein the FGFR inhibitor is selected from the group consisting of 3D-185, ABSK-011, ABSK-012, ABSK-061, ABSK-091, aldafermin, alofanib, AST-56100, AZD-4547, bemarituzumab, BFKB-8488A, BGS-2219, BIO-1262, BPI-17509, BPI-43487, CPL-304-110, derazantinib, E-7090, erdafitinib, EVER-4010001, EVT-601, FGF-401, fisogatinib, FPI-1966, futibatinib, gunagratinib, H3B-6527, HH-185, HMPL-453, HS-236, ICP-105, ICP-192, infigratinib, JAB-6000, KIN-3248, M-6123, MAX-40279, OM-RCA-001, pemigatinib, RLY-4008, rogaratinib, SAR-439115, SAR-442501, SC-0011, SY-4798, TT-00434, zoligratinib (FF-284), and WXSH-0011.

45. The method of claim 42 or 43, wherein the FGFR inhibitor is pemigatinib.

46. The method of claim 45, wherein the pemigatinib is administered to the subject at a dose of 13.5 mg PO daily or 9 PO mg or 4.5 mg PO daily and the dose is administered once daily for 14 days followed by 7 sequential days without administration of pemigatinib.

47. The method of claim 45 or claim 46, wherein the dose of Compound 1 is between about 20 to about 40 mg, about 40 to about 80 mg, about 80 to about 120 mg, about 120 mg to about 160 mg, or about 160 mg to about 200 mg.

48. The method of claim 42 or 43, wherein the FGFR inhibitor is futibatinib.

49. The method of claim 48, wherein the futibatinib is administered to the subject at a dose of 20 mg PO daily.

50. The method of claim 48 or 49, wherein the dose of Compound 1 is between about 20 to about 40 mg, about 40 to about 80 mg, about 80 to about 120 mg, about 120 mg to about 160 mg, or about 160 mg to about 200 mg.

51. The method of any one of claims 42-50, wherein the cancer is breast cancer, cholangiocarcinoma, esophageal cancer, head and neck squamous cell carcinoma, non-small cell lung cancer, stomach cancer, or subtype squamous cell carcinoma.

52. The method of claim 30 or claim 31, wherein the oncogene amplification comprises an amplification of CDK4, CDK6 or a combination thereof and wherein the treatment further comprises administering a CDK4/6 inhibitor.

53. The method of claim 52, wherein the CDK4/6 inhibitor is selected from the group consisting of abemaciclib, AG-122275, AM-5992, AT-7519, AU2-94, auceliciclib, BEBT-209, BPI-1178, BPI-16350, CS-3002, fascaplysin, FCN-437, FN-1501, GLR-2007, GW-491619, HEC-80797, HS-10342, IIIM-290, IIIM-985, lerociclib, milciclib maleate, MM-D37K, MS-140, NP-102, NUV-422, ON-123300, palbociclib, PF-06842874, PF-06873600, PF-07220060, QHRD-110, R-547, RGB-286199, RGT-419B, ribociclib, riviciclib, RO-0505124, SHR-6390, THR-53, THR-79, TQB-3303, TQB-3616, trilaciclib, TY-302, TY-302, voruciclib, VS2-370, WXWH-0240, XH-30002, and XZP-3287.

54. The method of claim 52, wherein the CDK4/6 inhibitor is abemaciclib.

55. The method of claim 54, wherein the abemaciclib is administered to the subject at a dose of about 50 mg twice daily, about 100 mg twice daily, or 150 mg twice daily.

56. The method of claim 54 or 55, wherein the dose of Compound 1 is between about 10 mg to about 20 mg, about 20 mg to about 40 mg, about 40 to about 80 mg, or about 80 to about 120 mg.

57. The method of any one of claims 52-56, wherein the cancer is esophageal cancer, non-small cell lung cancer, a sarcoma, or stomach cancer.

58. The method of any one of claims 6-57, wherein the therapeutic response comprises a reduction in the level of oncogene amplification in the tumor or tumor cells after treatment as compared to the level of oncogene amplification in the tumor or tumor cells prior to treatment.

59. The method of any of claims 1-58, further comprising obtaining a diagnostic indicator of oncogene amplification in a biological sample from the subject.

60. The method of claim 59, wherein the diagnostic indicator is obtained prior to a first administration of Compound 1.

61. The method of claim 59, wherein the diagnostic indicator is obtained subsequent to a first administration of Compound 1.

62. The method of claim 59, wherein the diagnostic indicator is obtained subsequent to multiple administrations of Compound 1.

63. The method of claim 59, wherein the diagnostic indicator results from a next generation sequencing (NGS)-based assay.

64. The method of claim 59, wherein the diagnostic indicator results from a fluorescence in situ hybridization (FISH) assay.

65. The method of claim 59, wherein the diagnostic indicator comprises an indicator for ecDNA-derived oncogene amplification.

66. The method of any one of claims 59-65, wherein the diagnostic indicator is obtained from tumor or liquid biopsy.

67. The method of any one of claims 1-66, wherein the method further comprises assessing a sample from a subject for the presence or level of one or more of a gene amplification, a focal gene amplification, ecDNA, HSR, or an ecDNA signature.

68. The method of any one of claims 1-66, wherein the method further comprises obtaining information of the presence or level of one or more of a gene amplification, a focal gene amplification, ecDNA, HSR, or an ecDNA signature in the tumor or tumor cells from the subject prior to, during or subsequent to the administration of Compound 1.
